SEQUENCE LISTING (24 sequences)

SEQ ID NO: 1 - DNA, 1080 bp, Human
  CDS (1)...(1080)
atgactctggagtccatcatggcgtgctgcctgagcgaggaggccaaggaagcccggcggatcaacgacgagatcgagcggcagctccgcagggacaagcgggacgcccgccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgccacagacaccgagaatatccgctttgtctttgctgccgtcaaggacaccatcctccagttgaacctgaaggagtacaatctggtctaa

SEQ ID NO: 2 - protein, 359 aa, Human
MTLESIMACCLSEEAKEARRINDEIERQLRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 3 - DNA, 1065 bp, Rat
  CDS (1)...(1065)
atgggctgcacactgagcgctgaggacaaggcggccgtggagcgcagcaagatgatcgaccgcaacctccgggaggacggagagaaggcagcgcgcgaggtcaagctgctgctgctgggtgctggtgaatccgggaagagcacaattgtgaagcagatgaaaattatccacgaggctggctactcagaggaagagtgtaagcagtacaaagcagtggtctacagcaacaccatccagtccatcattgccatcattagagccatggggagattgaaaatcgactttggagacgctgctcgtgcggatgatgctcgccaactcttcgtgcttgctggggctgcagaggaaggctttatgaccgcggagctcgccggcgtcataaagagactgtggaaggacagcggtgtgcaagcctgcttcaacagatcccgggagtaccagctgaacgattcggcggcgtactacctgaatgacttggacagaatagcacaaccaaattacatcccaacccagcaggatgttctcagaactagagtgaaaacgacgggaattgtggaaacccactttactttcaaagatcttcattttaaaatgtttgacgtgggaggccagagatcagagcggaagaagtggattcactgctttgaaggcgtgactgccatcatcttctgtgtggccctgagtgactatgacctggttcttgctgaggatgaagaaatgaaccggatgcatgaaagcatgaagctgttcgatagcatatgtaacaacaagtggtttacggacacatccatcatccttttcctgaacaagaaggacctcttcgaagagaagatcaaaaagagtcccctcacgatatgctatccagaatatgcaggctcaaacacatatgaagaggcggctgcgtatatccagtgtcagtttgaagacctcaataaaaggaaggacacaaaggaaatttacacccacttcacttgcgccacggatacgaagaatgtgcagtttgtgttcgatgctgtaacggacgtcatcataaagaataacctaaaagactgtggtctcttctaa

SEQ ID NO: 4 - protein, 354 aa, Rat
MGCTLSAEDKAAVERSKMIDRNLREDGEKAAREVKLLLLGAGESGKSTIVKQMKIIHEAGYSEEECKQYKAVVYSNTIQSIIAIIRAMGRLKIDFGDAARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYTHFTCATDTKNVQFVFDAVTDVIIKNNLKDCGLF

SEQ ID NO: 5 - DNA, 1080 bp, Mouse
  CDS (1)...(1080)
atgactctggagtccatcatggcgtgctgcctgagcgaggaggccaaggaagcccggaggatcaacgacgagatcgagcggcacgtgcgcagggacaagcgcgacgcccgccgggagctcaagctgctgctgctggggacaggggagagtggcaagagcaccttcatcaagcagatgaggatcatccacgggtcgggctactctgacgaagacaagcgcggcttcaccaagctggtgtatcagaacatcttcacggccatgcaggccatgatcagagcgatggacacgctcaagatcccatacaagtatgaacacaataaggctcatgcacaattggttcgagaggttgatgtggagaaggtgtctgcttttgagaatccatatgtagatgcaataaagagcttgtggaatgatcctggaatccaggagtgctacgacagacgacgggaatatcagttatctgactctaccaaatactatctgaatgacttggaccgtgtagccgacccttcctatctgcctacacaacaagacgtgcttagagttcgagtccccactacagggatcatcgaatacccctttgacttacaaagtgtcattttcagaatggtcgatgtagggggccaaaggtcagagagaagaaaatggatacactgctttgaaaatgtcacctccatcatgtttctagtagcgcttagcgaatatgatcaagttcttgtggagtcagacaatgagaaccgcatggaggagagcaaagcactctttagaacaattatcacctacccctggttccagaactcctctgtgattctgttcttaaacaagaaagatcttctagaggagaaaatcatgtattcccacctagtcgactacttcccagaatatgatggaccccagagagatgcccaggcagctcgagaattcatcctgaaaatgttcgtggacctgaaccccgacagtgacaaaatcatctactcccacttcacgtgcgccacagataccgagaacatccgcttcgtctttgcagccgtcaaggacaccatcctgcagctgaacctgaaggagtacaatctggtctaa

SEQ ID NO: 6 - protein, 359 aa, Mouse
MTLESIMACCLSEEAKEARRINDEIERHVRRDKRDARRELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPSYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTENIRFVFAAVKDTILQLNLKEYNLV

SEQ ID NO: 7 - DNA, 1053 bp, Bovine
  CDS (1)...(1053)
atgggggctggggccagcgctgaggagaagcactcaagggagctggaaaagaagctgaaagaagatgctgagaaagatgctcgaaccgtgaaactgctgcttctgggtgccggtgaatccgggaagagtaccattgtcaagcagatgaagattatccaccaggacgggtactcactggaagagtgtcttgagttcattgccatcatctatggcaacacgctacagtccatcctggccattgtgcgcgccatgaccacactcaacatccagtacggagactctgcgcgccaggacgacgcccgaaagctgatgcacatggcagacaccatcgaggagggcacgatgcccaaggagatgtcagacatcatccagcggctgtggaaggactccggtatccaggcctgtttcgaccgagcctcagagtaccagctcaacgactctgctggctactatctctcagacctggagcgcctggtaaccccgggctacgtgcccactgaacaggatgtgctgcgctcccgtgtcaagaccacgggtatcattgagacgcagttctccttcaaggacctcaactttcggatgttcgatgtgggcgggcagcgctcagagcgcaagaagtggatccactgcttcgagggggtgacctgcatcatcttcatcgcggcgctgagcgcctacgacatggtgctggtggaagacgacgaagtgaaccgcatgcacgagagcctgcacctgttcaacagtatctgcaaccaccgctacttcgccaccacgtccatcgtgctctttctcaacaagaaggacgtcttctcggagaagatcaaaaaggcgcaccttagtatctgctttccggactacaacgggcccaacacgtatgaggacgccggcaattacatcaaggtgcaattccttgagctcaacatgcgacgcgacgtgaaggagatctattcccacatgacatgcgccaccgacacgcagaacgtcaagtttgtcttcgacgctgtcaccgacatcatcatcaaggagaacctcaaagactgcgggctcttctga

SEQ ID NO: 8 - protein, 350 aa, Bovine
MGAGASAEEKHSRELEKKLKEDAEKDARTVKLLLLGAGESGKSTIVKQMKIIHQDGYSLEECLEFIAIIYGNTLQSILAIVRAMTTLNIQYGDSARQDDARKLMHMADTIEEGTMPKEMSDIIQRLWKDSGIQACFDRASEYQLNDSAGYYLSDLERLVTPGYVPTEQDVLRSRVKTTGIIETQFSFKDLNFRMFDVGGQRSERKKWIHCFEGVTCIIFIAALSAYDMVLVEDDEVNRMHESLHLFNSICNHRYFATTSIVLFLNKKDVFSEKIKKAHLSICFPDYNGPNTYEDAGNYIKVQFLELNMRRDVKEIYSHMTCATDTQNVKFVFDAVTDIIIKENLKDCGLF

SEQ ID NO: 9 - DNA, 1083 bp, Artificial Sequence
  Gilq6N3C alpha subunit
atgactctcgagtccatcatgggctgcacactgagcgctgaggacaaggcggccgtggagcgcagcaagatgatcgaccgcaacctccgggaggacggagagaaggcagcgcgcgaggtcaagctgctgctgctgggtgctggtgaatccgggaagagcacaattgtgaagcagatgaaaattatccacgaggctggctactcagaggaagagtgtaagcagtacaaagcagtggtctacagcaacaccatccagtccatcattgccatcattagagcnatggggagattgaaaatcgactttggagacgctgctcgtgcggatgatgctcgccaactcttcgtgcttgctggggctgcagaggaaggctttatgaccgcggagctcgccggcgtcataaagagactgtggaaggacagcggtgtgcaagcctgcttcaacagatcccgggagtaccagctgaacgattcggcggcgtactacctgaatgacttggacagaatagcacaaccaaattacatcccaacccagcaggatgttctcagaactagagtgaaaacgacgggaattgtggaaacccactttactttcaaagatcttcattttaaaatgtttgacgtgggaggccagagatcagagcggaagaagtggatccactgcttcgaaggcgtgactgccatcatcttctgtgtggccctgagtgactatgacctggttcttgctgaggatgaagaaatgaaccggatgcacgaaagcatgaagctgttcgatagcatatgtaacaacaagtggtttacggacacatccatcatccttttcctgaacaagaaggacctcttcgaagagaagatcaaaaagagtcccctcacgatatgctatccagaatatgcaggctcaaacacatatgaagaggcggctgcgtatatccagtgtcagtttgaagacctcaataaaaggaaggacacaaaggaaatttacacccacttcacttgcgccacggatacgaagaatgtgcagtttgtgttcgatgctgtaacggacgtcatcataaagaataacctaaaagactgtaatctcgtctga

SEQ ID NO: 10 - protein, 360 aa, Artificial Sequence
  Gilq6N3C alpha subunit
MTLESIMGCTLSAEDKAAVERSKMIDRNLREDGEKAAREVKLLLLGAGESGKSTIVKQMKIIHEAGYSEEECKQYKAVVYSNTIQSIIAIIRAMGRLKIDFGDAARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYTHFTCATDTKNVQFVFDAVTDVIIKNNLKDCNLV

SEQ ID NO: 11 - DNA, 1083 bp, Artificial Sequence
  Gilq6N35C alpha subunit
atgactctcgagtccatcatgggctgcacactgagcgctgaggacaaggcggccgtggagcgcagcaagatgatcgaccgcaacctccgggaggacggagagaaggcagcgcgcgaggtcaagctgctgctgctgggtgctggtgaatccgggaagagcacaattgtgaagcagatgaaaattatccacgaggctggctactcagaggaagagtgtaagcagtacaaagcagtggtctacagcaacaccatccagtccatcattgccatcattagagcnatggggagattgaaaatcgactttggagacgctgctcgtgcggatgatgctcgccaactcttcgtgcttgctggggctgcagaggaaggctttatgaccgcggagctcgccggcgtcataaagagactgtggaaggacagcggtgtgcaagcctgcttcaacagatcccgggagtaccagctgaacgattcggcggcgtactacctgaatgacttggacagaatagcacaaccaaattacatcccaacccagcaggatgttctcagaactagagtgaaaacgacgggaattgtggaaacccactttactttcaaagatcttcattttaaaatgtttgacgtgggaggccagagatcagagcggaagaagtggatccactgctttgaaggcgtgactgccatcatcttctgtgtggccctgagtgactatgacctggttcttgctgaggatgaagaaatgaaccggatgcatgaaagcatgaagctgttcgatagcatatgtaacaacaagtggtttacggacacatccatcatccttttcctgaacaagaaggacctcttcgaagagaagatcaaaaagagtcccctcacgatatgctatccagaatatgcaggctcaaacacatatgaagaggcggctgcgtatatccagtgtcagtttgaagacctcaataaaaggaaggacacaaaggaaatttactcccacttcacgtgcgccacagacaccgagaatatccgctttgtctttgctgccgtcaaggacaccatcctccagttgaacctgaaggagtacaatctggtctaa

SEQ ID NO: 12 - protein, 360 aa, Artificial Sequence
  Gilq6N35C alpha subunit
MTLESIMGCTLSAEDKAAVERSKMIDRNLREDGEKAAREVKLLLLGAGESGKSTIVKQMKIIHEAGYSEEECKQYKAVVYSNTIQSIIAIIRAMGRLKIDFGDAARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYSHFTCATDTENIRFVFAAVKDTILQLNLKEYNLV

SEQ ID NO: 13 - DNA, 1083 bp, Artificial Sequence
  Gilq37N3C alpha subunit
atgactctcgagtccatcatggcgtgctgcctgagcgaggaggccaaggaagcccggcggatcaacgacgagatcgagcggcagctccgcagggacaagcgggacgcacgtcgtgaggtcaagctgctgctgctgggtgctggtgaatccgggaagagcacaattgtgaagcagatgaaaattatccacgaggctggctactcagaggaagagtgtaagcagtacaaagcagtggtctacagcaacaccatccagtccatcattgccatcattagagctatggggagattgaaaatcgactttggagacgctgctcgtgcggatgatgctcgccaactcttcgtgcttgctggggctgcagaggaaggctttatgaccgcggagctcgccggcgtcataaagagactgtggaaggacagcggtgtgcaagcctgcttcaacagatcccgggagtaccagctgaacgattcggcggcgtactacctgaatgacttggacagaatagcacaaccaaattacatcccaacccagcaggatgttctcagaactagagtgaaaacgacgggaattgtggaaacccactttactttcaaagatcttcattttaaaatgtttgacgtgggaggccagagatcagagcggaagaagtggatccactgcttcgaaggcgtgactgccatcatcttctgtgtggccctgagtgactatgacctggttcttgctgaggatgaagaaatgaaccggatgcacgaaagcatgaagctgttcgatagcatatgtaacaacaagtggtttacggacacatccatcatccttttcctgaacaagaaggacctcttcgaagagaagatcaaaaagagtcccctcacgatatgctatccagaatatgcaggctcaaacacatatgaagaggcggctgcgtatatccagtgtcagtttgaagacctcaataaaaggaaggacacaaaggaaatttacacccacttcacttgcgccacggatacgaagaatgtgcagtttgtgttcgatgctgtaacggacgtcatcataaagaataacctaaaagactgtaatctcgtctga

SEQ ID NO: 14 - protein, 360 aa, Artificial Sequence
  Gilq37N3C alpha subunit
MTLESIMACCLSEEAKEARRINDEIERQLRRDKRDARREVKLLLLGAGESGKSTIVKQMKIIHEAGYSEEECKQYKAVVYSNTIQSIIAIIRAMGRLKIDFGDAARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYTHFTCATDTKNVQFVFDAVTDVIIKNNLKDCNLV

SEQ ID NO: 15 - DNA, 1083 bp, Artificial Sequence
  Gilq37N35C alpha subunit
atgactctcgagtccatcatggcgtgctgcctgagcgaggaggccaaggaagcccggcggatcaacgacgagatcgagcggcagctccgcagggacaagcgggacgcacgtcgtgaggtcaagctgctgctgctgggtgctggtgaatccgggaagagcacaattgtgaagcagatgaaaattatccacgaggctggctactcagaggaagagtgtaagcagtacaaagcagtggtctacagcaacaccatccagtccatcattgccatcattagagcnatggggagattgaaaatcgactttggagacgctgctcgtgcggatgatgctcgccaactcttcgtgcttgctggggctgcagaggaaggctttatgaccgcggagctcgccggcgtcataaagagactgtggaaggacagcggtgtgcaagcctgcttcaacagatcccgggagtaccagctgaacgattcggcggcgtactacctgaatgacttggacagaatagcacaaccaaattacatcccaacccagcaggatgttctcagaactagagtgaaaacgacgggaattgtggaaacccactttactttcaaagatcttcattttaaaatgtttgacgtgggaggccagagatcagagcggaagaagtggatccactgctttgaaggcgtgactgccatcatcttctgtgtggccctgagtgactatgacctggttcttgctgaggatgaagaaatgaaccggatgcatgaaagcatgaagctgttcgatagcatatgtaacaacaagtggtttacggacacatccatcatccttttcctgaacaagaaggacctcttcgaagagaagatcaaaaagagtcccctcacgatatgctatccagaatatgcaggctcaaacacatatgaagaggcggctgcgtatatccagtgtcagtttgaagacctcaataaaaggaaggacacaaaggaaatttactcccacttcacgtgcgccacagacaccgagaatatccgctttgtctttgctgccgtcaaggacaccatcctccagttgaacctgaaggagtacaatctggtctaa

SEQ ID NO: 16 - protein, 360 aa, Artificial Sequence
  Gilq37N35C alpha subunit
MTLESIMACCLSEEAKEARRINDEIERQLRRDKRDARREVKLLLLGAGESGKSTIVKQMKIIHEAGYSEEECKQYKAVVYSNTIQSIIAIIRAMGRLKIDFGDAARADDARQLFVLAGAAEEGFMTAELAGVIKRLWKDSGVQACFNRSREYQLNDSAAYYLNDLDRIAQPNYIPTQQDVLRTRVKTTGIVETHFTFKDLHFKMFDVGGQRSERKKWIHCFEGVTAIIFCVALSDYDLVLAEDEEMNRMHESMKLFDSICNNKWFTDTSIILFLNKKDLFEEKIKKSPLTICYPEYAGSNTYEEAAAYIQCQFEDLNKRKDTKEIYSHFTCATDTENIRFVFAAVKDTILQLNLKEYNLV

SEQ ID NO: 17 - DNA, 1062 bp, Artificial Sequence
  Gqil131N25C alpha subunit
atgggctgcacactgagcgctgaggacaaggcggccgtggagcgcagcaagatgatcgaccgcaacctccgggaggacggagagaaggcagcccgggagctcaagctgctgctgctcgggacaggagagagtggcaagagtacgtttatcaagcagatgagaatcatccatgggtcaggatactctgatgaagataaaaggggcttcaccaagctggtgtatcagaacatcttcacggccatgcaggccatgatcagagccatggacacactcaagatcccatacaagtatgagcacaataaggctcatgcacaattagttcgagaagttgatgtggagaaggtgtctgcttttgagaatccatatgtagatgcaataaagagtttatggaatgatcctggaatccaggaatgctatgatagacgacgagaatatcaattatctgactctaccaaatactatcttaatgacttggaccgcgtagctgaccctgcctacctgcctacgcaacaagatgtgcttagagttcgagtccccaccacagggatcatcgaatacccctttgacttacaaagtgtcattttcagaatggtcgatgtagggggccaaaggtcagagagaagaaaatggatacactgctttgaaaatgtcacctctatcatgtttctagtagcgcttagtgaatatgatcaagttctcgtggagtcagacaatgagaaccgaatggaggaaagcaaggctctctttagaacaattatcacatacccctggttccagaactcctcggttattctgttcttaaacaagaaagatcttctagaggagaaaatcatgtattcccatctagtcgactacttcccagaatatgatggaccccagagagatgcccaggcagcccgagaattcattctgaagatgttcgtggacctgaacccagacagtgacaaaattatctactcccacttcacgtgcgcaacggatacgaagaatgtgcagtttgtgttcgatgctgtaacggacgtcatcataaagaataacctaaaagactgtggtctcttctaa

SEQ ID NO: 18 - protein, 353 aa, Artificial Sequence
  Gqil131N25C alpha subunit
MGCTLSAEDKAAVERSKMIDRNLREDGEKAARELKLLLLGTGESGKSTFIKQMRIIHGSGYSDEDKRGFTKLVYQNIFTAMQAMIRAMDTLKIPYKYEHNKAHAQLVREVDVEKVSAFENPYVDAIKSLWNDPGIQECYDRRREYQLSDSTKYYLNDLDRVADPAYLPTQQDVLRVRVPTTGIIEYPFDLQSVIFRMVDVGGQRSERRKWIHCFENVTSIMFLVALSEYDQVLVESDNENRMEESKALFRTIITYPWFQNSSVILFLNKKDLLEEKIMYSHLVDYFPEYDGPQRDAQAAREFILKMFVDLNPDSDKIIYSHFTCATDTKNVQFVFDAVTDVIIKNNLKDCGLF

SEQ ID NO: 19 - DNA, 6 bp, Artificial Sequence
  mutation of GilQ6N
ctcgag

SEQ ID NO: 20 - DNA, 6 bp, Artificial Sequence
  mutation of GilQ6N
ctggag

SEQ ID NO: 21 - DNA, 44 bp, Mouse
gatccatgactctcgagtccatgtactgagagctcaggtagtac

SEQ ID NO: 22 - DNA, 54 bp, Artificial Sequence
  duplex linker
aattccatggatgactctcgagtccatggtacctactgagagctcaggtagtac

SEQ ID NO: 23 - DNA, 36 bp, Artificial Sequence
  duplex linker
gatctccatggcccgggtaggtaccgggcccagatc

SEQ ID NO: 24 - DNA, 32 bp, Artificial Sequence
  duplex linker
agctgtatctagatagcatagatctatcttaa

(19) United States
(12) Patent Application Publication
(10) Pub. No.: US 2004/0072157 A1
(43) Pub. Date: Apr. 15, 2004

(54) Soluble chimeric G protein alpha subunits

(76) Inventor: Stephen G. Graber, Morgantown, WV (US)

Correspondence Address: BURNS DOANE SWECKER & MATHIS L L P, POST OFFICE BOX 1404, ALEXANDRIA, VA, 22313-1404, US

(21) Appl. No.: 10/059,266

(22) Filed: Jan. 31, 2002

Related U.S. Application Data
(60) Provisional application No. 60/265,068, filed on Jan. 31, 2001.

(51) Int. Cl. C12Q 1/68; C07H 21/04; C12P 21/02; C12N 5/06; C07K 14/705

(52) U.S. Cl. 435/6; 435/69.1; 435/320.1; 435/325; 530/350; 536/23.5

(57) ABSTRACT

The present invention provides a chimeric α subunit of G proteins having n (n=3 to 50) amino acids from the N-terminus and c (c=3 to 50) amino acids from the C-terminus of a donor α subunit of a G protein at the N-terminus and C-terminus of the chimeric α subunit, wherein the internal part of the chimeric α subunit is a recipient α subunit which is the α subunit of a G protein of a different subclass than the donor α subunit having m amino acids and/or d amino acids optionally removed from the N-terminus and C-terminus, respectively, of the recipient α subunit, wherein m is n−10, n−9, n−8, n−7, n−6, n−5, n−4, n−3, n−2, n−1, n, n+1, n+2, n+3, n+4, n+5, n+6, n+7, n+8, n+9 or n+10 and d is c-10, c-9, c-8, c-7, c-6, c-5, c-4, c-3, c-2, c-1, c, c+1, c+2, c+3, c+4, c+5, c+6, c+7, c+8, c+9 or c+10.

This application claims the benefit of U.S. Provisional Application, No. 60/265,068, filed on Jan. 31, 2001.

### FIELD OF THE INVENTION

The present invention is related to chimeric G protein α subunits having unique properties in affecting receptor coupling of the G proteins. Some of the chimeric G protein α subunits of the present invention are soluble so that they can be manipulated relatively easily.

### BACKGROUND OF THE INVENTION

Overview of G Protein Mediated Signaling

A large number of different G protein coupled receptors (GPCR) exist in mammals to mediate a diverse array of physiological responses initiated by hormones, neurotransmitters, sensory stimuli and other signaling molecules. This family of receptors is thought to share a structural arrangement of seven transmembrane segments joined by extracellular and intracellular loops that couple external signals to cellular responses via a family of heterotrimeric G proteins. Each heterotrimeric G protein is comprised of an α subunit and a βγ dimer that serve as functional units. Agonist binding to a GPCR catalyzes the exchange of GDP for GTP on the α subunit which releases the G protein from the receptor and dissociates the α and βγ subunits which subsequently modulate the activities of their target effectors. The α subunit has an intrinsic GTPase activity that hydrolyzes GTP to GDP and plays a role in terminating the signal initiated by agonist binding to the receptor. While not all combinations are allowed, some 20 α, 6 β, and 12 γ subunits form a large number of distinct G protein heterotrimers. Nevertheless, G proteins are usually classified by the nature of their α subunit into Gs, Gi, Gq or G12/13 families. Each GPCR is coupled to a heterotrimeric G protein that is critical for transmitting the signal initiated by receptor binding to the intracellular environment. While some GPCRs are capable of coupling with members of all four G protein families, in general, an individual GPCR couples with a limited number of G proteins from a single family, and individual G proteins modulate the activities of a limited number of distinct effectors [1].

In the heart, muscarinic receptors play a critical role in regulating the force and rate of cardiac contraction. Muscarinic receptors are typical GPCRs which in human include 5 molecularly distinct members: M1, M2, M3, M4 and M5. M1, M3 and M5 couple preferentially with Gq heterotrimers, i.e. heterotrimeric G proteins having Gq α subunits. In contrast, M2 and M4 couple preferentially with Gi heterotrimers.

Mechanisms of Signaling Selectivity

An enormous body of work investigating mechanisms underlying selectivity in G protein coupled signaling pathways now exists. Certainly the early view that signaling selectivity would manifest itself on the basis of specific protein interactions allowing a receptor to couple with a unique G protein to modulate a single effector is no longer tenable with the accumulating evidence of a network of interactions that converge and diverge at multiple levels. Indeed, it is now clear that understanding signaling selectivity will ultimately require consideration of entities distinct from the comparatively simple receptor to G protein to effector axes that have occupied much of our attention heretofore. Increasing evidence suggesting that receptor oligomerization may play an important role in GPCR activation have begun to emerge. Although it has not been demonstrated that such events are required for G protein activation, numerous receptors have been shown to participate in oligomerization and the functional and structural aspects of oligomerization have become widespread enough to be reviewed [2]. It has been clear for some time that families of G protein receptor kinases (GRKs) and arrestins regulate one form of receptor desensitization [3] and that these processes exhibit considerable selectivity [4]. The observation that the GRKs may themselves be regulated by PKC [5] suggests yet other regulatory inputs. An additional layer of complexity has been added by the discovery that GRK phosphorylation of GPCRs plays a role in switching the receptor from G protein dependent pathways to those traditionally associated with tyrosine kinase receptors [6,7]. A role for c-Src in GPCR coupled activation of mitogen-activated protein kinases (MAPK) has been demonstrated [8] and recently it has been suggested that arrestin can function as a scaffolding protein in the recruitment of c-SRC to a GPCR containing complex [9]. GRKs are also implicated in GPCR mediated cytoskeletal rearrangements [7]. GPCR involvement with SH2 domain signaling proteins, small GTP-binding proteins, PDZ domain containing proteins and polyproline-binding proteins makes a strong case for the suggestion that individual GPCRs will activate both G protein-dependent and G protein-independent pathways, and that GPCRs may function analogously to tyrosine kinase receptors in forming heterologous signaling complexes through a plethora of protein binding domains [7]. Yet another protein family that may modify GPCR function are the recently described receptor activity modifying proteins (RAMPS) [10].

Similarly, G proteins may have their activities modulated by an increasing array of accessory proteins. Descriptions of a family of RGS (regulators of G protein signaling) proteins capable of attenuating the modulation of effector activities by Gα subunits [11,12] and the growing understanding that once again subtype diversity contributes to the establishment of signaling selectivity [13-15] suggests yet another point at which selectivity is achieved in a complicated, subtype dependent manner. Perhaps most confounding of all is the recent description of a novel family of proteins that function as receptor independent activators of G protein signaling (AGS proteins) [16,17]. One family member, AGS3, inhibits GDP dissociation from Giα family members, and blocks both rhodopsin activation of transducin and 5-HT_{1B} receptor coupling to Gi, and may serve to activate Gβγ dependent pathways in a receptor independent fashion [18,19]. AGS3 is detected in various tissues, and a portion of AGS3 exists as a complex with Giα in the cell [20]. Another family member, AGS1, is a Ras-related G protein that functions as a guanine nucleotide exchange factor for Giα [21]. These and related proteins will certainly define novel and unexpected control points for G protein mediated signaling pathways.

While multiple approaches continue to discern various mechanisms contributing to selectivity in these pathways, several generalizations regarding signaling selectivity in a cellular context have become clear:

Individual GPCRs may signal via both G protein dependent and independent pathways.

There are a variety of receptor independent mechanisms that modulate G protein activities.

There are numerous points at which cross-talk among G-protein dependent and independent pathways occur.

As individual aspects of these complex interactions are appreciated, it will likely become possible to understand entire pathways at a cellular, or even higher, level. At present, the mechanism by which an agonist occupied GPCR activates it's cognate heterotrimeric G protein is unknown, as is the molecular basis for the selectivity with which individual GPCRs couple with their cognate G proteins. While this selectivity ranges among individual GPCRs from the completely promiscuous to the exquisitely selective, understanding the molecular basis by which it is achieved (or not achieved) is important in understanding signal transduction. Considering that G-protein pathways control essential functions in all tissues and are ubiquitous within the animal kingdom, elucidation of the basic mechanisms controlling the receptor-G protein interaction will contribute to understanding cellular function and dysfunction in many systems and can lead to new therapeutic modalities in treating numerous disease states.

Receptor-G Protein Interface

Elucidation of the crystal structures of several α subunits in both active and inactive conformations [22-25], an isolated βγ subunit [26], and a complete heterotrimer [27,28] has begun to define a mechanistic basis for a wealth of data from mutagenesis and chimera and peptide studies defining functional domains on G protein subunits.

The C-terminus of α subunits plays a critical role in mediating receptor G protein selectivity. Synthetic peptides from the C-terminus of αt (340-350) have been shown to stabilize the active conformation of metarhodopsin II [29,30], while alanine scanning mutagenesis of the same region has identified four specific residues crucial for at activation by rhodopsin [31]. Similarly, two C-terminal peptides from αs (354-372 and 384-394), but not the corresponding peptides from αi2, could evoke high affinity agonist binding to adrenergic receptors and block their ability to activate αs [32]. Substitution of as few as 3-5 C-terminal amino acids from αi½ for the corresponding residues in aq allowed several receptors normally signaling exclusively through αi subunits to activate the chimeric α subunits and stimulate PLC-β [33,34]. Recently these studies have been extended and refined to show that similar C-terminal chimeras allow receptors normally coupling with either as or aq to change selectivity depending on the nature of the C-terminal residues in the chimera, and that residues at positions −3, −4 and −5 were critical determinants of this selectivity [35-37]. Significantly, exceptions to the generality of these C-terminal residues as determinants of coupling selectivity were noted [35-37]. Recent work suggests that while the incorrect C-terminal context is sufficient to prevent receptor coupling, the correct C-terminal context is not sufficient to allow coupling when presented in the context of an otherwise inappropriate α subunit [38,39]. Differences in the approaches taken (transfection vs. reconstitution) may explain the differences among these studies, however, a recent reconstitution study demonstrated that the 5 C-terminal amino acids of transducin are sufficient to allow Gsα to couple with rhodopsin [40].

N-Terminus of Alphα subunits

The N-terminus of α subunits has also received considerable attention. Early studies indicated N-terminal regions of α subunits were required for interactions with rhodopsin [41], mastoporan, a wasp venom thought to mimic receptor activation of G proteins [42] and α2-adrenergic receptors [43]. Replacement of amino acids 1-210 of αi1 with those from αt impaired but did not prevent coupling with 5-HT_{1B} receptors [44]. While changes in either one of a pair of cysteines at positions 9 and 10 of αq was well tolerated with respect to M1 muscarinic receptor coupling, both A and S double mutants impaired receptor coupling, as did removal of the 6 amino acid extension unique to the αq family [45]. Wess and coworkers found that removal of the unique αq extension did not significantly alter coupling to receptors normally coupled to the aq family while it allowed coupling to several receptors normally coupled to either αi/o or αs families and suggested that the αq extension is critical for constraining receptor coupling [46]. An addition of the unique 6 amino acid αq extension to αi1 was not sufficient to prevent coupling to the M2 muscarinic receptor which gained coupling to αq when the extension was removed [46].

Additional regions of α subunits have been shown to be critical for selective interactions with specific receptors. Chimeric α subunits revealed that sequences in addition to the C-terminus were required for specificity of activation of α16 subunits by the C5a receptor [47]. The α4-helix and α4-β6 loop region has been shown to be important for at interactions with rhodopsin [48-50]. Work from the inventor's laboratory, in collaboration with Heidi Hamm's laboratory, revealed that the α4-helix and α4-β6 loop region mediates the discrimination between αi/o and αt subunits by 5HT₁ receptors [44] and Q304 and E308 have been identified as primarily responsible [51]. While the βγ dimer is clearly required for activation of Gα by receptors [52,53] and direct interactions of βγ subunits with receptors have been demonstrated [54,56], relatively few reports have suggested selectivity in receptor recognition of G protein heterotrimers based on the composition of the βγ dimer. Several reports have recently demonstrated such selectivity for several GPCRs, largely on the basis of the α subunit or its prenyl modification [57-60]. This fits well with the proposed sequential two-site mechanism of signal transfer from rhodopsin to transducin involving specific recognition of conformationally distinct sites on R* by Gtα(340-350) and Gtγ(50-71)farnesyl [61].

Structural elements of receptors involved in functional interactions with G proteins have been identified in an overwhelming number of studies involving the use of synthetic peptides, chimeric substitutions, scanning mutagenesis and other mutational approaches. These studies identified the second- and third- intracellular loops of GPCRs as essential for selectivity and functional coupling with G proteins. The muscarinic receptors are of particular relevance to the present invention. Work from the Wess and Brann laboratoris identified residues crucial for selective G protein coupling in the N-terminal portion of the third intracellular loop adjacent to transmembrane domain 5 [62-65] and in the C-terminal portion of the third intracellular loop adjacent to transmembrane domain 6 [34,66,67]. Random mutagenesis through the second intracellular loop of the M5 muscarinic receptor has shown that one face of a proposed α-helix is involved in maintaining the inactive ground state of the receptor while the opposite face is involved in G protein coupling [68]. A role for IC3 in determining selectivity of G protein coupling and for IC2 in activating G proteins was clearly demonstrated with both loss of function and gain of function using chimeric receptors involving loop replacements of various GPCRs (including M1 and M2 muscarinic receptors) into rhodopsin [69]. The first crystal structure of a GPCR (rod cell rhodopsin) has recently been solved at 2.8 Å resolution [70] and will allow critical evaluation of this enormous body of structure-function studies in rhodopsin and other GPCRs. The extensive network of interhelical interactions revealed in the ground state of rhodopsin [70] supports the notion that GPCRs are constrained in an inactive state and that disruption of the constraints by agonist binding induces movement of the transmembrane helices leading to receptor activation [71-73]. However, despite the recent progress in structural determinations, the molecular mechanism of signal transfer from GPCR to G protein remains unknown. The crystal structure of rhodopsin does not provide direct information about either the structure of the activated state of the receptor (R*), or the dynamics of the transition between the two states. Furthermore, rhodopsin is unique among GPCRs, possessing greater efficiency and less constitutive activity than other GPCRs. The covalently attached chromophore (cis-retinal) serves as an “inverse agonist” in the absence of light and photo-isomerizes to an agonist in the presence of light, allowing rapid activation of the receptor even in the absence of transducin [74]. While non-covalently attached trans-retinal is capable of activating rhodopsin, it is far more effective when covalently attached [75].

Affinity Shift Assays and Receptor Activation Models

The high affinity agonist binding state of a GPCR is the ternary complex of receptor and guanine nucleotide free G protein heterotrimer. In membranes where expressed receptors are either in excess of, or unable to couple with, endogenous G proteins, receptor-G protein coupling may be studied by the addition of purified, exogenous G proteins. By measuring the increase in high affinity agonist binding to the receptor of interest in the presence of exogenous wildtype or chimeric G proteins, one can determine whether the mutations introduced into the G protein affect receptor-G protein coupling. This assay is based on early work with native receptors [76,77] and has been completely described for several recombinant receptors [78,79]. The affinity shift assay requires that partially purified membranes containing the expressed receptor be reconstituted with exogenous G proteins. A radioligand binding assay using a low concentration of agonist (near the high-affinity K_{D} of the receptor such that little or no binding occurs to uncoupled receptors) is used to determine the enhancement of binding due to the exogenous G proteins. Thus, the functional interaction between receptor and G protein is measured directly by radioligand binding, eliminating interference from other proteins that may modulate the activity of the downstream effector or of the activated Gα subunit. Also, the stoichiometry of receptors and G proteins can be tightly regulated which allows for detection of relatively subtle changes in their functional interaction resulting from either differing affinities of a receptor for a particular G protein or from a change in the agonist affinity of a particular ternary complex.

If it is difficult to express the receptor in excess of the endogenous G proteins, the endogenous G proteins may be removed by urea treatment of the membrane preparation [80]. Treatment with urea will remove proteins not tightly associated with the membranes. Although experiments with GTPγS reveal that a small portion of the endogenous G proteins remain after the urea stripping, this treatment does significantly improve affinity shift activity. The G protein concentration is another important consideration in setting up an affinity shift assay with a particular receptor. The G proteins must be present at saturating concentrations with respect to the receptor in order to ensure maximum binding at the concentration of agonist chosen. Because different receptors have distinct affinities for G proteins ([78] and FIG. 5), the G protein saturation point needs to be determined for each receptor examined. Use of saturating amounts of G protein allows meaningful comparisons among activities resulting from reconstitutions with native or chimeric Gα subunits.

In principle, the magnitude of the affinity shift will depend on both receptor number and the difference between the high and low affinity K_{D} values for a given receptor. In order to obtain a measurable affinity shift, the expressed receptors must be in excess of any endogenous G proteins capable of coupling with the receptor. As the number of uncoupled receptors increases, the magnitude of the shift will also increase. The difference between high and low affinity K_{D} values also varies among GPCRs, such that the magnitude of the affinity shift may vary among receptors even when expressed at comparable levels. These phenomena must be taken into consideration when comparing affinity shift activities among different receptors or receptor preparations with significantly different expression levels. When making such comparisons affinity shift activities must be normalized. Affinity shift activity is calculated as the ratio of specific binding activity in the presence of exogenous G protein (reconstituted membranes) to the specific binding activity in non-reconstituted control membranes. The normalized affinity shift (NAS) can be expressed as the ratio of the difference between binding in chimera reconstituted membranes (CRM) and non-reconstituted membranes (M) to the difference between binding in native G-protein reconstituted membranes (NGRM) and non-reconstituted membranes (M). This may be summarized by the following relationship:

NAS=[CRM−M]÷[NGRM−M]

When normalized affinity shift is calculated, native or chimeric G proteins that do not interact with a given receptor will have normalized activities of zero, while fully active G proteins will have activities of one. Generally, an appropriate native G protein will produce the largest affinity shift activity with a given receptor, though occasionally chimeric G proteins with enhanced coupling properties have been observed. Such chimeras have normalized affinity shifts significantly greater than one.

Currently, the modified ternary complex model (also known as the two-state model) is the most widely accepted model for the activation of GPCRs [81,82]. According to the model, a GPCR exists in equilibrium between inactive (R) and active (R*) conformations. The R* conformation is stabilized by guanine nucleotide-free heterotrimeric G proteins and binds agonists with the highest affinity. Agonists thereby shift the equilibrium toward the accumulation of R*. However, receptors are capable of adopting the R* conformation in the absence of agonists giving rise to “constitutive” activity and agonist independent activation of G proteins and signaling cascades. Characterization of the present invention is based on both G protein modulation of receptor properties in affinity shift assays [44,51] and agonist occupied receptor modulation of G protein properties in agonist stimulated guanine nucleotide exchange assays. While thermodynamic considerations dictate that there must be negative heterotropic effects on the affinities of the receptor for agonist and of the G protein for guanine nucleotide, analysis of chimeras in both assays may identify domains that contribute differentially to these effects (i.e. by changes in the kinetics or rate limiting step in guanine nucleotide exchange). To date there has been strong correlation between results from affinity shift and agonist stimulated GTP exchange assays . It is becoming clear that the currently accepted two-state model does not adequately explain the observed behavior of GPCRs, especially the existence of multiple activated states of many receptors [83]. In this regard, a strength of biochemical reconstitution is that it may provide mechanistic insights not apparent from studies in intact cells. One of the uses of the chimeric α subunits of the present invention is to form along with with a β subunit and a γ subunit a heterotrimeric G protein, which can be used to reconstitute membranes having endogenous G proteins previously removed. The reconstituted membrane can be used in receptor coupling studies, in which the specificity of the receptor coupling is controlled by a donor G protein α subunit which contributes the N- and C-terminal amino acids to a recipient G protein α subunits in the formation of the chimeric α subunit of the present invention. These receptor coupling studies are useful in screening for active agonists or antagonists of specific GPCRs, which can lead to the identification of new pharmaceutical agents.

Elucidation of the basic mechanisms controlling these pathways will contribute to the understanding of cellular function and dysfunction in many systems and can lead to new therapeutic modalities in treating numerous disease states. At the biochemical level, significant contributions in this area can be made by developing the ability to employ components of signaling pathways in reconstitution paradigms. The ability to functionally couple receptors expressed in Sf9 cell membranes with exogenous G proteins has been developed and are used to examine the G protein coupling behavior of distinct subtypes of receptors capable of functionally distinguishing among individual Gα subunits. The molecular basis of this selectivity is defined by comparing the abilities of G protein heterotrimers containing chimeric α subunits, comprised of various regions of αi1, αt and αq subunits, to interact with individual receptors. Functional interactions were assessed by examining both the ability of the G protein to induce the high affinity state of the receptor and the ability of the agonist occupied receptor to catalyze guanine nucleotide exchange on the G protein. A strength of the present invention is that the chimeric α subunits of the invention allow examinations of the interactions of defined molecular species in a single eukaryotic membrane environment using a reconstitution paradigm where the stoichiometries can be controlled with some precision. The present invention shows that multiple and distinct determinants of selectivity exist for various receptor families. Identification of the individual amino acids on Gα subunits involved in functional interactions with receptors would allow understanding the selective interaction of individual GPCRs with particular Gα subunits as well as the precise molecular mechanism underlying receptor mediation of the guanine nucleotide exchange process, the initial intracellular step in a profusion of signal transduction cascades.

Direct coupling assays previously showed that switching the C-terminal portions (up to 35 amino acids) is not sufficient to switch receptor coupling and that addition of the 6 N-terminal amino acids of Gqα to Gi1α does not prevent receptor coupling. However, within the scope of the present invention is the discovery that the receptor coupling properties of a G protein is mainly dependent on the amino acid sequences in the N-terminus and the C-terminus of the G protein's α subunit.

### SUMMARY OF THE INVENTION

According to the present invention, converting the α subunit of a recipient G protein of a certain class, e.g. one of Gq, Gs, G1 or G12/13, into a chimeric protein subunit by

(a) replacing a number, m, of consecutive amino acids in the N-terminus and a number, d, of consecutive amino acids in the C-terminus of the α subunit of the recipient G protein with a number, n, of consecutive amino acids from the N-terminus and a number, c, of consecutive amino acids from the C-terminus, respectively, of the α subunit of a donor G protein of a different class, e.g. one of Gq, Gs, G1 or G12/13 not the same as the recipient G protein; wherein n and c are independently about 3 to about 50, about 3 to about 44, about 3 to about 42, about 3 to about 40, about 3 to about 38, about 3 to about 35, about 6 to about 50, about 6 to about 44, about 6 to about 42, about 6 to about 40, about 6 to about 38, about 6 to about 35, about 6 to about 32, about 6 to about 30, about 8 to about 38, about 8 to about 35, about 8 to about 32, about 8 to about 30, about 10 to about 32, about 10 to about 30, about 10 to about 28, about 10 to about 25, about 12 to about 25 or about 12 to about 20 amino acids; m is equal to n−10, n−9, n−8, n−7, n−6, n−5, n−4, n−3, n−2, n−1, n, n+1, n+2, n+3, n+4, n+5, n+6, n+6, n+7, n+8, n+9 or n+10; d is equal to c−10, c−9, c−8, c−7, c−6, c−5, c−4, c−3, c−2, c−1, c, c+1, c+2, c+3, c+4, c+5, c+6, c+7, c+8, c+9 or c+10; and n and c can be the same or different;

(b) replacing a number, m, of consecutive amino acids in the N-terminus of the α subunit of the recipient G protein with a number, n, of consecutive amino acids from the N-terminus of the α subunit of a donor G protein of a different class, e.g. one of Gq, Gs, Gi or G12/13 not the same as the recipient G protein; and adding a number, c, of consecutive amino acids from the C-terminus of the α subunit of the donor G protein to the C-terminus of the α subunit of the recipient G protein; wherein n and c are independently about 3 to about 50, about 3 to about 44, about 3 to about 42, about 3 to about 40, about 3 to about 38, about 3 to about 35, about 6 to about 50, about 6 to about 44, about 6 to about 42, about 6 to about 40, about 6 to about 38, about 6 to about 35, about 6 to about 32, about 6 to about 30, about 8 to about 38, about 8 to about 35, about 8 to about 32, about 8 to about 30, about 10 to about 32, about 10 to about 30, about 10 to about 28, about 10 to about 25, about 12 to about 25 or about 12 to about 20 amino acids; m is equal to n−10, n−9, n−8, n−7, n−6, n-S, n−4, n−3, n−2, n−1, n, n+1, n+2, n+3, n+4, n+5, n+6, n+6, n+7, n+8, n+9 or n+10; d is equal to c−10, c−9, c−8, c−7, c−6, c−5, c−4, c−3, c−2, c−1, c, c+1, c+2, c+3, c+4, c+5, c+6, c+7, c+8, c+9 or c+10; and n and c can be the same or different;

(c) replacing a number, d, of consecutive amino acids in the C-terminus of the α subunit of the recipient G protein with a number, c, of consecutive amino acids from the C-terminus of the α subunit of a donor G protein of a different class, e.g. one of Gq, Gs, G1 or G12/13 not the same as the recipient G protein; and adding a number, i.e. n, of consecutive amino acids from the N-terminus of the α subunit of the donor G protein to the N-terminus of the α subunit of the recipient G protein; wherein n and c are independently about 3 to about 50, about 3 to about 44, about 3 to about 42, about 3 to about 40, about 3 to about 38, about 3 to about 35, about 6 to about 50, about 6 to about 44, about 6 to about 42, about 6 to about 40, about 6 to about 38, about 6 to about 35, about 6 to about 32, about 6 to about 30, about 8 to about 38, about 8 to about 35, about 8 to about 32, about 8 to about 30, about 10 to about 32, about 10 to about 30, about 10 to about 28, about 10 to about 25, about 12 to about 25 or about 12 to about 20 amino acids; d is equal to is equal to c−10, c−9, c−8, c−7, c−6, c−5, c−4, c−3, c−2, c−1, c, c+1, c+2, c+3, c+4, c+5, c+6, c+7, c+8, c+9 or c+10; and n and c can be the same or different; or

(d) adding a number, n, of consecutive amino acids from the N-terminus and a number, c, of consecutive amino acids from the C-terminus of the α subunit of the donor G protein to the N-terminus and C-terminus, respectively, of the α subunit of the recipient G protein; wherein n and c are independently about 3 to about 50, about 3 to about 44, about 3 to about 42, about 3 to about 40, about 3 to about 38, about 3 to about 35, about 6 to about 50, about 6 to about 44, about 6 to about 42, about 6 to about 40, about 6 to about 38, about 6 to about 35, about 6 to about 32, about 6 to about 30, about 8 to about 38, about 8 to about 35, about 8 to about 32, about 8 to about 30, about 10 to about 32, about 10 to about 30, about 10 to about 28, about 10 to about 25, about 12 to about 25 or about 12 to about 20 amino acids; and n and c can be the same or different;

creates a chimeric G protein having the receptor coupling selectivity of the donor G protein (wherein the n and c consecutive amino acids are counted by starting from the ending amino acid at the N-terminus and C-terminus, respectively, of the α subunit of the donor G protein). In other words, the receptor coupling properties of the donor G protein can be transferred to the recipient G protein if an appropriate portion of the N-terminus of the donor G protein and an appropriate portion of the C-terminus of the donor G protein are transferred to the recipient G protein by amino acid replacements, amino acid additions, or a mixture thereof described above. Examples of n and c independently include 3, 6, 25, 31, 35 and 37, preferably 6, 35 and 37.

The present invention also shows that adding 1 to 30, or 2 to 30, preferably 2 to 20 (e.g. 3 or 15), more preferably 4, 5, 6, 7, 8, 9 or 10, most preferably 6, N-terminal amino acids of Gqα to Gi1α together with replacing 2 to 45, preferably 3 to 40 (e.g. 3, 18, 24 or 40), more preferably 4 to 35 (e.g. 5, 10, 15, 20, 25, 30 or 35), also preferably 5 to 25 or 5 to 11 (e.g. 11), C-terminal amino acids of Gi1α with those from Gqα creates a chimeric G protein α subunit (e.g. Gi1q6N35C) that couples M1 receptors (M1 receptors normally are coupled to G proteins having Gqα subunits) and does not couple M2 receptors (M2 receptors normally are coupled to G proteins having Giα subunits).

The present invention provides a chimeric α subunit of G proteins, which chimeric α subunit is represented by formula (I):

B₁-B₂-B₃ (I),

wherein

B₁ is the N-terminus and B₃ is the C-terminus of the chimeric α subunit;

B₁ is a peptide having the N-terminal amino acid sequence of n amino acids in length from donor alpha, wherein donor alpha is an α subunit of a donor G protein;

B₃ is a peptide having the C-terminal amino acid sequence of c amino acids in length from donor alpha;

B₂ is selected from the group consisting of (A) recipient alpha, which is an α subunit of a recipient G protein different from the α subunit of the donor G protein, (B) recipient alpha minus n−10, n−9, n−8, n−7, n−6, n−5, n−4, n−3, n−2, n−1, n, n+1, n+2, n+3, n+4, n+5, n+6, n+7, n+8, n+9 or n+10 consecutive amino acid residues from the N-terminus, (C) recipient alpha minus c−10, c−9, c−8, c−7, c−6, c−5, c−4, c−3, c−2, c−1, c, c+1, c+2, c+3, c+4, c+5, c+6, c+7, c+8, c+9 or c+10 consecutive amino acid residues from the C-terminus, and (D) recipient alpha minus n−10, n−9, n−8, n−7, n−6, n−5, n−4, n−3, n−2, n−1, n, n+1, n+2, n+3, n+4, n+5, n+6, n+7, n+8, n+9 or n+10 consecutive amino acid residues from the N-terminus and minus c−10, c−9, c−8, c−7, c−6, c−5, c−4, c−3, c−2, c−1, c, c+1, c+2, c+3, c+4, c+5, c+6, c+7, c+8, c+9 or c+10 amino acid residues from the C-terminus; wherein

n and c are independently about 3 to about 50, about 3 to about 44, about 3 to about 40, about 3 to about 38, about 3 to about 35, about 6 to about 50, about 6 to about 44, about 6 to about 40, about 6 to about 38, about 6 to about 35, about 6 to about 32, about 6 to about 30, about 8 to about 38, about 8 to about 35, about 8 to about 32, about 8 to about 30, about 10 to about 32, about 10 to about 30, about 10 to about 28, about 10 to about 25, about 12 to about 25 or about 12 to about 20 consecutive amino acid residues;

n and c can be the same or different; and

B₁ and B₂, and B₂ and B₃, are linked with a peptide bond.

Within the scope of the present invention is a chimeric G protein comprising a subunit of a G protein, a γ subunit of a G protein and a chimeric α subunit of the present invention, wherein the β subunit and γ subunit are from the same or different G protein.

The present invention also provides a DNA comprising a nucleotide sequence encoding the chimeric α subunit represented by formula (I).

### BRIEF DESCRIPTION OF DRAWINGS

FIG. 1. Secondary structure of some chimeric Gα subunits containing various regions of Gtα, Gi1α and Gqα subunits. Other than q6N and q6N35C chimeras, the other chimeras depicted in FIG. 1 were known in the prior art. For the chimeras made with Gtα and Gi1α, amino acids 10 through 13 in Gi1α have no corresponding amino acids in Gtα, so amino acid 9 in Gtα corresponds to amino acid 9 in Gi1α, but amino acid 10 of Gtα corresponds to amino acid 14 of Gi1α. As a result, Chi2 was made by linking amino acids 1-298 of Gi1α with amino acids 295-350 of Gtα. For the chimeras made with Gqα and Gi1α, it should be noted that Gqα has a unique 6 amino acid N-terminal extension, so amino acids 1-6 of Gqα have no corresponding amino acid sequence in Gi1α and amino acid 7 of Gqα corresponds to amino acid 1 of Gi1α. There are no gaps in the C-terminus such that amino acids 325-359 of Gqα correspond directly to amino acids 320-354 of Gi1α. In FIG. 1, numbers above the chimeric structures indicate the junction points of Gtα and Gi1α sequences and refer to the amino acid positions in Gtα. Numbers for the wild type forms of Gtα, Gi1α and Gqα represent the total numbers of their amino acid residues. The diagram at the bottom of FIG. 1 depicts the secondary structural domains common to Gα subunits.

FIG. 2. Effects of urea stripping on M4 coupling. M4 receptors (˜150 fmol) were reconstituted with or without a 100 fold excess of Gi1 and incubated with 5 nM Oxo-M in the presence of the indicated additional components. Data presented were the means±SD of triplicates from a representative experment.

FIG. 3. Effects of urea stripping on M1 coupling. M1 receptors (˜100 finol) were reconstituted with or without a 100 fold excess of Gq or Gi1 and incubated with 5 nM Oxo-M. Data presented were the means±SD of triplicates from a representative experment.

FIG. 4. Affinity shift activity of Gα subunits with M1 and M2 receptors. Affinity shift activities represents the -fold enhancement above buffer controls of high affinity [³H-]-Oxo-M binding in membranes expressing the indicated muscarinic receptor reconstituted with a 100 fold excess of G protein heterotrimers containing the indicated Gα subunit. Data presented were the means±SEM from 2-4 experiments where [Oxo-M] was ˜5 nM.

FIG. 5. Concentration dependence of Gi1 in affinity shift assays for individual Gi1-coupled receptors. Sf9 cell membranes expressing the indicated Gi1-coupled receptors were reconstituted with increasing concentrations of Gi1 heterotrimer. Normalized affinity shift activities from 3 to 4 independent experments for each receptor were fit to a single-site interaction between receptor and G protein. Saturation was achieved for each receptor. However, for visual purposes, the curves have been extended to a common endpoint.

FIG. 6. Functional coupling of receptors to the indicated Gi1/Gt chimeras. Sf9 cell membranes expressing individual receptors were reconstituted with the indicated chimeric Gα and βγ subunits. Data represent the normalized affinity shift activities as mean±SEM from 3 to 9 independent experiments for each receptor. Exogenous G proteins were present in 40-400 fold molar excess over receptors during reconstitution to achieve the maximal specific binding during the binding assays. The results show that the α4 helix-α4/β6 region of Gi1 is important for specific recognition between Gi1 and the serotonin 5-HT_{1A}, 5-HT_{1B}, or muscarinic M2 receptors but not the adenosine A1 receptor.

FIG. 7. Functional coupling of receptors to the indicated Gi1/Gq chimeras. Sf9 cell membranes expressing individual receptors were reconstituted with the indicated chimeric Gα and βγ subunits. Data represent the normalized affinity shift activities as mean±SEM from 3 to 9 independent experiments for each receptor. Exogenous G proteins were present in 40-400 fold molar excess over receptors during reconstitution to achieve the maximal specific binding during the binding assays. The results show that the C-terminal residues of Gi1α are important for its interactions with the 5-HT_{1A}, 5-HT_{1B}, muscarinic M2 and adenosine A1 receptors. However, the receptors differ in their sensitivities to the number of C terminal amino acid residues that are replaced.

FIG. 8 shows the muscarinic receptor-catalyzed GDP/GTP exchange on Gα subunits. GDP/GTP exchange was measured as GTPγS binding in membranes expressing the indicated muscarinic receptor (0.8 nM final) reconstituted with a 60 fold excess of G protein heterotrimers containing the indicated Gα subunits. Bars represent the fold-enhancement by agonist above basal nucleotide exchange in the absence of agonist on Gα subunits and are the mean±SEM from 3-5 experiments. The asterisks above the bars indicate values significantly different than 1.0 (p<0.05, one-sample t-test) and the absence of error bars indicates that there was no significant difference between basal and agonist driven rates in any experiment. The inset graph depicts moles of GTPγS bound per mole of receptor in membranes expressing M2 muscarinic receptors with or without reconstitution with Gi1 in the presence and absence of 2 μM Oxo-M as indicated. The lines are least squares regression lines and the slopes represent the rate of GTPγS binding. Rates of GTPγS binding to all Gα subunits were determined as shown in the inset graph and the mean basal rates of GTPγS binding (mol GTPγS/mol receptor/min) were as follows—Gi1, 0.21; Q6N, 0.21; Q35C, 0.09; Q6N35C, 0.10; Gqi5C, 0.05; Gq, 0.10. The basal rate for Gi1 was significantly greater than all other subunits except Q6N (p<0.05, Dunnett's Multiple Comparison Test).

FIG. 9 shows the nucleotide sequence (SEQ ID NO:1) of the DNA encoding human Gq α subunit and its amino acid sequence (SEQ ID NO:2) with Genbank Accession No. U40038.

FIG. 10 shows the nucleotide sequence of the DNA (SEQ ID NO:3) encoding rat Gi1 α subunit and its amino acid sequence (SEQ ID NO:4) with Genbank Accession No. M17527.

FIG. 11 shows the nucleotide sequence (SEQ ID NO:5) of the DNA encoding mouse Gq α subunit and its amino acid sequence (SEQ ID NO:6) with Genbank Accession No. M55412.

FIG. 12 shows the nucleotide sequence (SEQ ID NO:7) of the DNA encoding bovine Gt α subunit and its amino acid sequence (SEQ ID NO:8) with Genbank Accession No. M11115.

FIG. 13 shows the nucleotide sequence (SEQ ID NO:9) of the DNA encoding Gi1q6N3C α subunit and its amino acid sequence (SEQ ID NO:10). The Gilq6N3C α subunit was prepared by adding 6 amino acid residues from the N-terminus of human Gq a subunit to the N-terminus of the rat Gi1 α subunit and replacing 3 amino acid residues of the C-terminus of the rat Gi1 α subunit with 3 amino acid residues from the C-terminus of human Gq α subunit.

FIG. 14 shows the nucleotide sequence (SEQ ID NO:11) of the DNA encoding Gi1q6N35C α subunit and its amino acid sequence (SEQ ID NO:12). The Gi1q6N35C α subunit was prepared by adding 6 amino acid residues from the N-terminus of human Gq α subunit to the N-terminus of the rat Gi1 α subunit and replacing 35 amino acid residues of the C-terminus of the rat Gi1 α subunit with 35 amino acid residues from the C-terminus of human Gq α subunit.

FIG. 15 shows the nucleotide sequence (SEQ ID NO:13) of the DNA encoding Gi1q37N3C α subunit and its amino acid sequence (SEQ ID NO:14). The Gi1q37N3C α subunit was prepared by replacing 31 amino acid residues of the N-terminus of the rat Gi1 α subunit with 37 amino acid residues from the N-terminus of human Gq α subunit and replacing 3 amino acid residues of the C-terminus of the rat Gi1 α subunit with 3 amino acid residues from the C-terminus of human Gq α subunit.

FIG. 16 shows the nucleotide sequence (SEQ ID NO:15) of the DNA encoding Gi1q37N35C α subunit and its amino acid sequence (SEQ ID NO:16). The Gi1q37N35C α subunit was prepared by replacing 31 amino acid residues of the N-terminus of the rat Gi1 α subunit with 37 amino acid residues from the N-terminus of human Gq α subunit and replacing 35 amino acid residues of the C-terminus of the rat Gi1 α subunit with 35 amino acid residues from the C-terminus of human Gq α subunit.

FIG. 17 shows the nucleotide sequence (SEQ ID NO:17) of the DNA encoding Gqil31N25C α subunit and its amino acid sequence (SEQ ID NO:18). The Gqil31N25C α subunit was prepared by replacing 37 amino acid residues of the N-terminus of the human Gq α subunit with 31 amino acid residues from the N-terminus of the rat Gi1 α subunit and replacing 25 amino acid residues of the C-terminus of the human Gq α subunit with 25 amino acid residues from the C-terminus of the rat Gi1 α subunit.

### DETAILED DESCRIPTION OF THE INVENTION

The present invention also provides the chimeric α subunit of G proteins represented by formula (I), wherein n and c independently are about 6 to about 50, about 6 to about 44, about 6 to about 40, about 6 to about 38, about 6 to about 35, about 6 to about 32, about 6 to about 30, about 8 to about 38, about 8 to about 35, about 8 to about 32, about 8 to about 30, about 10 to about 32, about 10 to about 30, about 10 to about 28, about 10 to about 25, about 12 to about 25, or about 12 to about 20.

The present invention also provides the chimeric α subunit of G proteins represented by formula (I), wherein n and c independently are about 6 to about 44, about 6 to about 40, about 6 to about 38, about 6 to about 35, about 6 to about 32, about 6 to about 30, about 8 to about 38, about 8 to about 35, about 8 to about 32, about 8 to about 30, about 10 to about 32, about 10 to about 30, about 10 to about 28, about 10 to about 25, about 12 to about 25, or about 12.

Moreover, the present invention provides a chimeric α subunit of formula (I), wherein n and c independently are about 6 to about 40, about 6 to about 38, about 6 to about 35, about 6 to about 32, about 6 to about 30, about 8 to about 38, about 8 to about 35, about 8 to about 32, about 8 to about 30, about 10 to about 32, about 10 to about 30, about 10 to about 28, about 10 to about 25, about 12 to about 25, or about 12 to about 20.

In addition, the present invention provides the chimeric α subunit of G proteins represented by formula (I), wherein n and c independently are about 6 to about 38, about 6 to about 35, about 6 to about 32, about 6 to about 30, about 8 to about 38, about 8 to about 35, about 8 to about 32, about 8 to about 30, about 10 to about 32, about 10 to about 30, about 10 to about 28, or about 10 to about 25.

The present invention also provides the chimeric α subunit of G proteins represented by formula (I), wherein n and c independently are about 6 to about 40, about 6 to about 38, about 6 to about 35, about 6 to about 32, or about 6 to about 30.

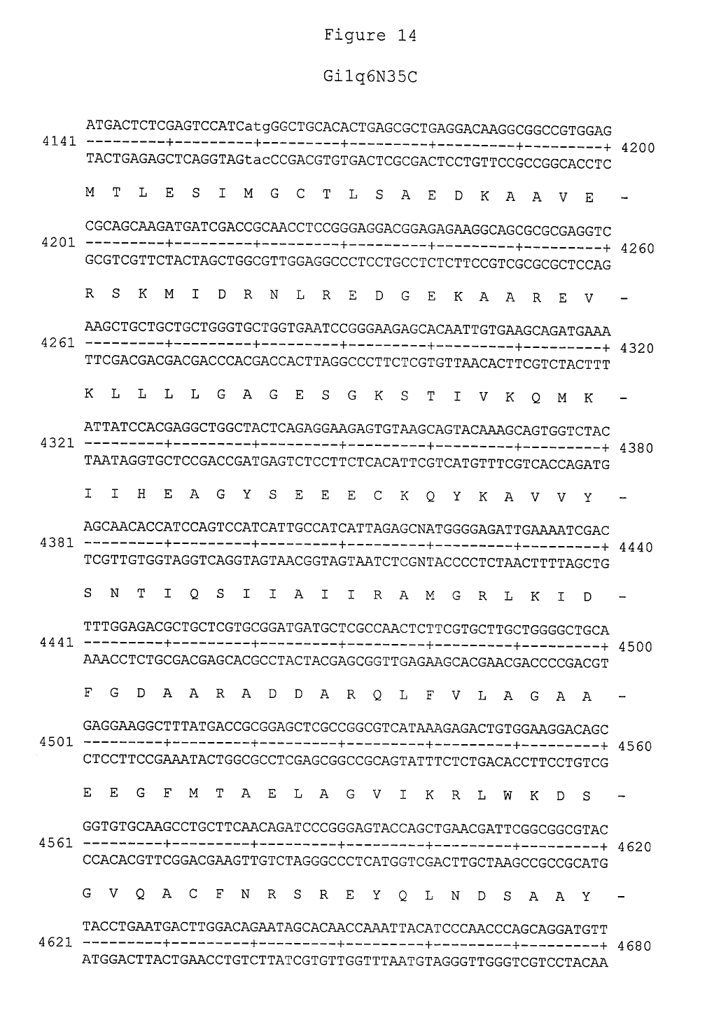

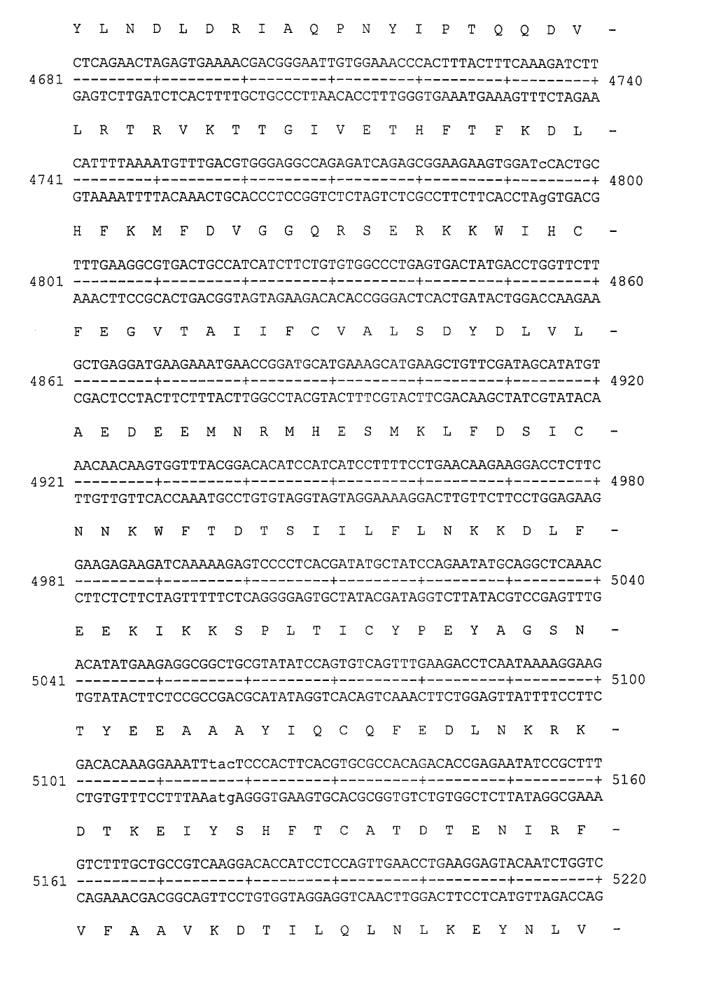

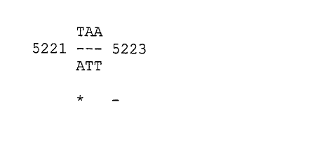

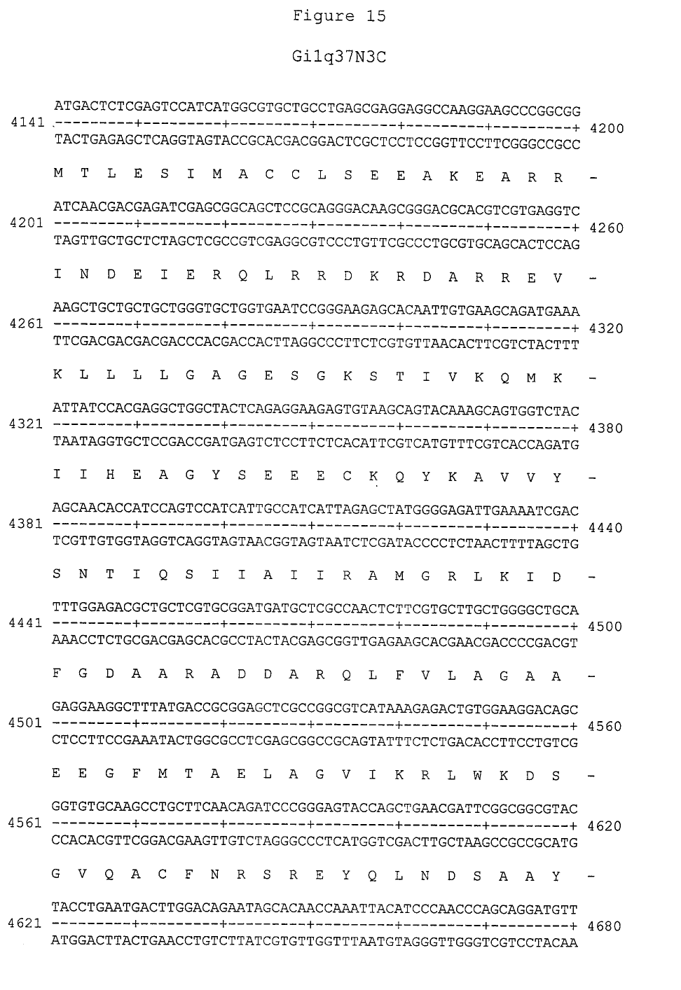

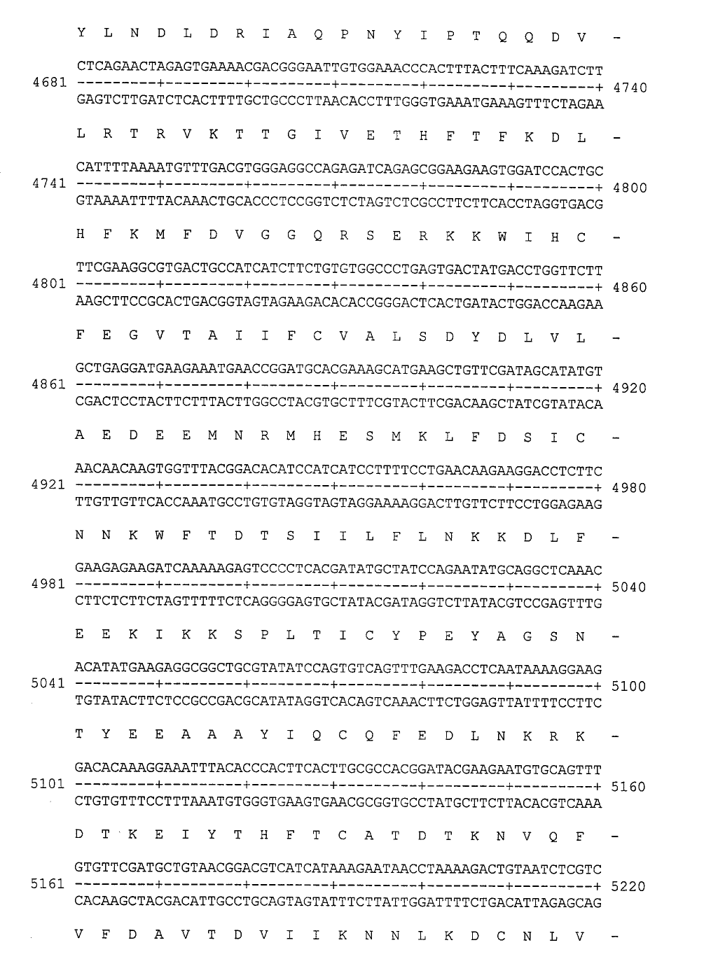

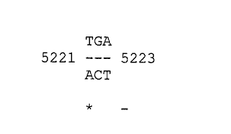

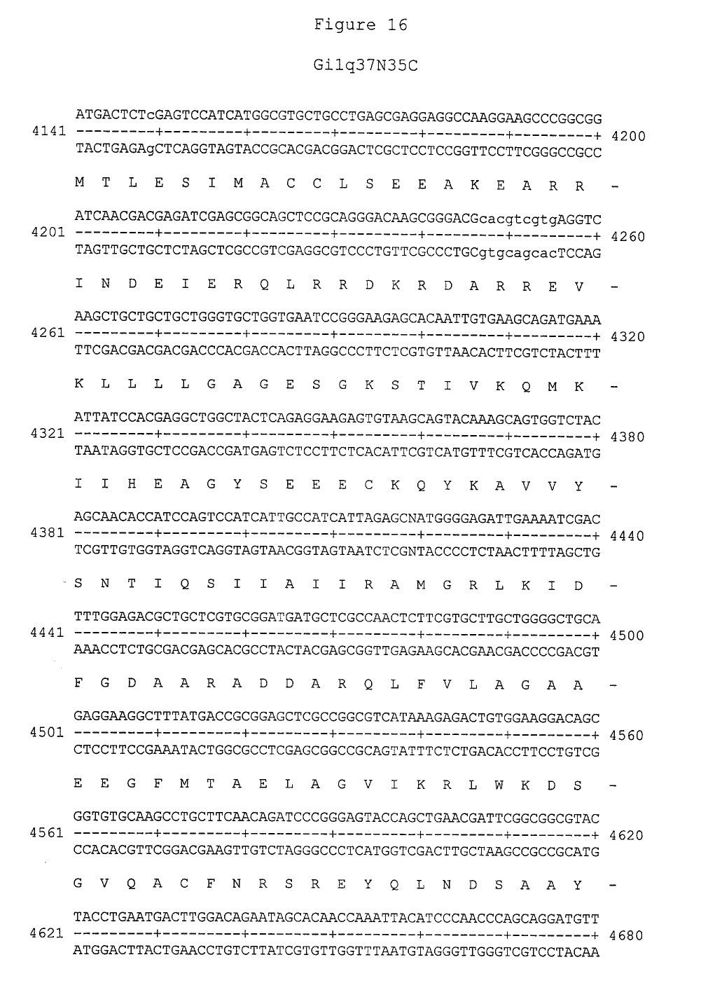

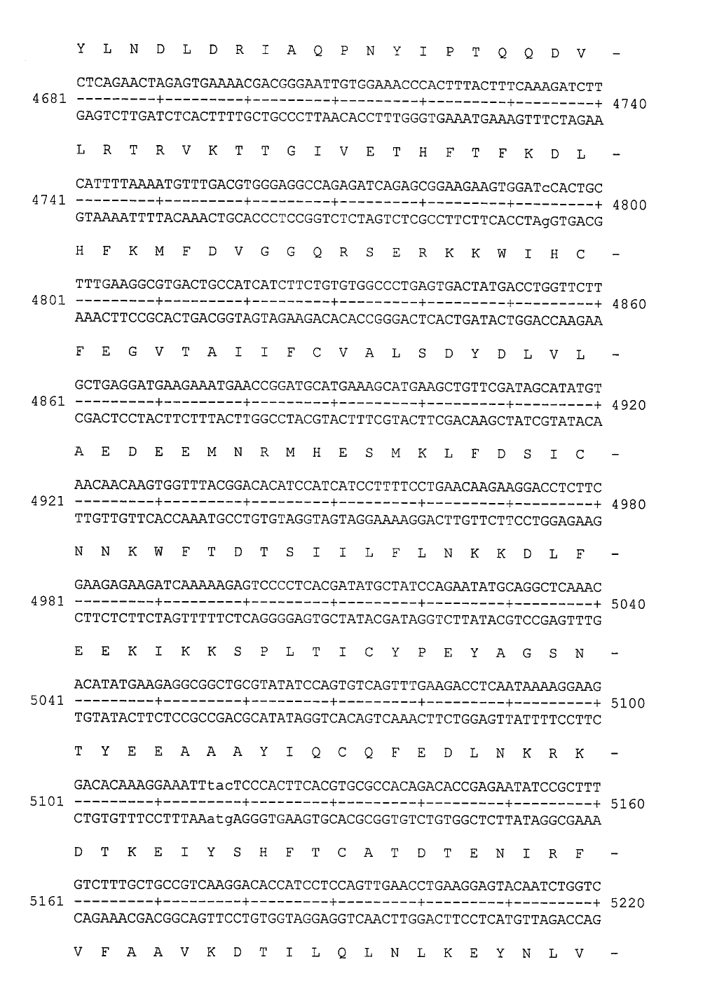

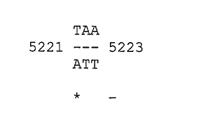

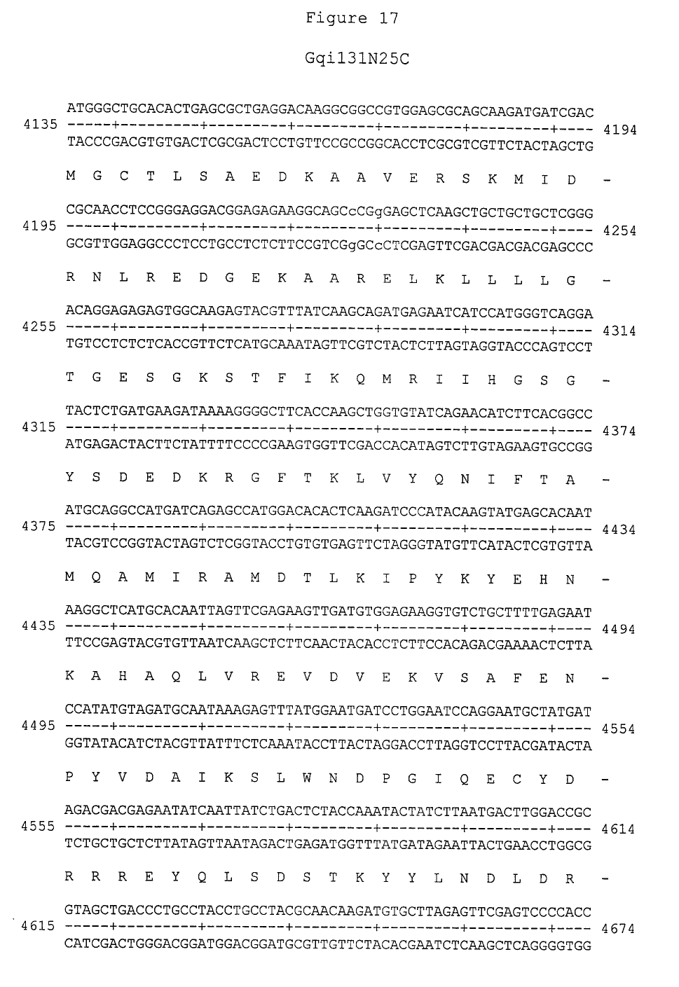

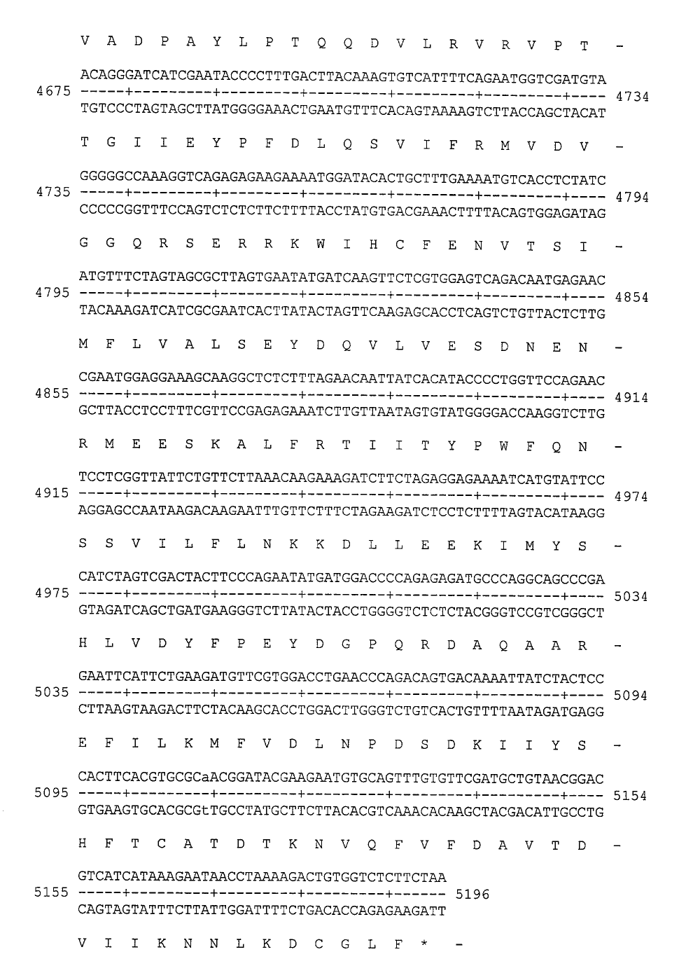

Within the scope of the present invention is the chimeric α subunit of G proteins represented by formula (I), wherein n and c independently are about 6, about 31, about 35 or about 37 amino acids. For instance, the present invention also provides the following chimeric α subunits: Gi1q6N35C, Gi1q37N35C and Gqi131N25C described in FIGS. 14, 16 and 17.

The present invention also provides chimeric α subunits of formula (I) wherein donor alpha is a Gq α subunit and recipient alpha is a Gi1 α subunit.

The present invention also provides chimeric α subunits of formula (I) wherein recipient alpha is a Gq α subunit and donor alpha is a Gi1 α subunit.

The present invention also provides chimeric α subunits of formula (1) wherein donor alpha is a Gq α subunit and recipient alpha is a Gs α subunit.

The present invention also provides chimeric α subunits of formula (I) wherein donor alpha is a Gq α subunit and recipient alpha is a G12/13 α subunit.

Within the scope of the present invention is a protein having an amino acid sequence with 99, 98 or 95% identity with SEQ ID NO:12, 16 or 18, wherein when said protein is combined with a β subunit and a γ subunit of G proteins, the resultant heterotrimeric protein has a receptor coupling specificity similar to that of a heterotrimer formed by the chimeric α subunit represented by SEQ ID NO:12, 16 or 18 and the β subunit and the γ subunit.

One of the main discoveries of the present invention is that the context of both the N- and C-terminal parts of Gα subunits plays a major role in determining the receptor coupling selectivity of a G protein. Therefore, replacing the N-terminal and C-terminal portions of Gqα with the corresponding regions of Gi1α (e.g. Gi1α amino acids 1-30 and 320-354) will produce a chimeric protein that couples receptors normally coupled by Gi proteins rather than receptors normally coupled by Gq proteins. A similar strategy of making other chimeric α subunits would switch receptor coupling selectivity in the Gs or G15/16 families. Adding more Gqα N-terminal amino acid sequence will improve the coupling of Gi1q6N35C. For instance, replacing some of amino acids 1 through 30 of the Gi1α portion of the Gilq6N35C chimera with a corresponding consecutive number of amino acids from amino acids 7 through 37 of Gqα subunit results in another chimeric protein with better receptor coupling than the G protein having the Gi1q6N35C chimera. As few as 3 to 11 C-terminal amino acids are required for maximal receptor coupling in the presence of the appropriate N-terminal sequences. Instead of Gi1α, other Giα subunits, e.g. Gi2α or Gi3α, can be used in making the chimeric α subunits of the present invention.

In making the chimeric α subunits of the present invention, one skilled in the art can rely on the amino acid sequences for the Gtα, Giα (e.g. Gi1α, Gi2α or Gi3α), Gqα, Gsα and/or G15/16α subunits known in the art. The nucleotide sequences, and amino acid sequences obtainable therefrom, disclosed in prior art references are hereby incorporated by reference. For instance, the amino acid sequence for bovine transducin α subunit, Gtα, can be obtained from the cDNA sequence disclosed in Genbank Accession #M11115 (Nature, vol. 315, pp. 242-245, 1985); the amino acid sequence for rat Gi1α subunit can be obtained from Genbank Accession #17527 (J. Biol. Chem., vol. 262, pp. 14241-14249, 1987); and similarly, the amino acid sequence for mouse Gqα subunit can be obtained from Genbank #M55412 (Proc. Natl. Acad. Sci. USA, vol. 87, pp. 9113-9117, 1990).

The chimeric G protein α subunits of the present invention can be combined with any βγ dimer of G proteins to form a heterotrimeric G protein having a chimeric α subunit. The receptor coupling selectivity of the resulting heterotrimeric G protein is mainly determined by the chimeric α subunit. For instance, in the examples of heterotrimeric G proteins actually prepared in the present application, the chimeric α subunits prepared based on the bovine Gtα, rat Gi1α and mouse Gqα subunits were combined with β1 and γ2 subunits. The inventor has also found that combining the chimeric α subunits prepared based on the amino acid sequences of the rat Gi1α and mouse Gqα subunits with β1 and γ1 subunits, retinal βγ dimers, or brain βγ dimers formed heterotrimeric G proteins that had receptor coupling selectivity mainly dependent on the N-terminus and C-terminus of the donor G protein's α subunits of the chimera.

The chimeric proteins of the present invention can be made using donor G proteins and recipient G proteins from different species.

Another aspect of the present invention is a DNA comprising a nucleotide sequence that encodes any of the chimeric α subunits of G proteins of the present invention. All the nucleotide sequences of the DNA's that encode any of the chimeric α subunits of the present invention based on the degeneracy of the genetic code are within the scope of the present invention. Examples of the DNA of the present invention include DNAs comprising a nucleotide sequence of SEQ ID NO:11, 15 or 17. Within the scope of the invention is a DNA comprising a nucleotide sequence which can hybridize with the nucleotide sequence represented by SEQ ID NO:11, 15 or 17 at a stringency condition of 42° C., 0.2×SSC and 0.1% SDS or a more stringent condition of 68° C., 0. 1×SSC and 0.1% SDS.

Also within the scope of the present invention is a method of producing the purified chimeric α subunit of a G protein, said method comprising the following steps:

(1) replacing appropriate amino acids from the N-terminus of the α subunit of the recipient G protein with appropriate amino acids from the N-terminus of the α subunit of a donor G protein or inserting appropriate amino acids from the N-terminus of the α subunit of the donor G protein to the N-terminus of the α subunit of the recipient G protein;

(2) replacing appropriate amino acids from the C-terminus of the α subunit of the recipient G protein with appropriate amino acids from the C-terminus of the α subunit of a donor G protein or inserting appropriate amino acids from the C-terminus of the α subunit of the donor G protein to the C-terminus of the α subunit of the recipient G protein to obtain a chimeric α subunit of a G protein (note that steps (1) and (2) can be reversed or performed simultaneously); and

(3) optionally isolating the resulting chimeric α subunit.

Another method of producing the purified chimeric α subunit of a G protein, comprises the steps of

(1) joining appropriate DNA sequences encoding the N-terminus and C-terminus of the α subunit of the donor G protein with appropriate DNA sequences from a DNA encoding the α subunit of the recipient G protein and/or changing the DNA sequences encoding the N-terminus and C-terminus of the α subunit of the recipient G protein in a gene encoding the recipient G protein's α subunit to obtain a chimeric DNA so that the chimeric DNA encodes an α subunit having the N-terminal and C-terminal amino acid sequences of the α subunit of a donor G protein;

(2) linking a 5′ end of the chimeric DNA with a 3′ end of a promoter region to obtain a DNA construct;

(3) transcribing the DNA construct to obtain a mRNA; and

(4) translating the mRNA to obtain the chimeric α subunit.

Still another method of producing the purified chimeric α subunit of a G protein, comprises the steps of joining or changing appropriate regions of mRNA which encode the appropriate N-terminal and C-terminal regions of the chimeric α subunit of the G protein to obtain a mRNA construct, and translating mRNA construct to obtain the chimeric α subunit.

Another aspect of the present invention are derivatives of the chimeric α subunit described above that have useful signaling properties with respect to their receptor interactions and abilities to modulate downstream effector activities similar to the chimeric α subunit described above.

Also within the scope of the present invention is a method of using the chimeric α subunits of the present invention to form heterotrimeric G proteins and using the resulting heterotrimeric G proteins in receptor coupling or receptor affinity shift assays. Another aspect of the present invention is to use this method to identify potential agonists or antagonists of receptors.

The selectivity inherent in G protein mediated signal transduction pathways ultimately involves a network of interactions that converge and diverge at multiple levels and vary depending upon the cellular context examined. Nevertheless, the determinants of functional coupling at the comparatively simple receptor-G protein interface remain to be appreciated at the molecular level. Critical domains on G protein α, β and γ subunits as well as portions of the intracellular loops of various G protein coupled receptors (GPCRs) have been identified with a variety of experimental approaches. Systematic work by the inventor's lab and others has shown that even closely related GPCRs differ significantly in the basic parameters underlying functional interactions with their cognate G proteins. Thus, despite nearly identical structures and clearly conserved elements in the basic mechanisms, individual receptor-G protein interfaces must be examined in detail to elucidate the precise molecular interactions defining the mechanism by which an agonist occupied GPCR initiates the transduction of a signal across a cell membrane.

According to the present invention,(i) individual GPCRs require multiple and distinct domains on G protein α subunits for functional interactions and (ii) different combinations of these domains are used to achieve selectivity with specific GPCRs. A part of the present invention focuses on defining the molecular mechanisms responsible for the G protein coupling selectivity among muscarinic receptors, and shows the involvement of the domains identified with a panel of both closely and distantly related GPCRs. Using both gain of function and loss of function experiments allows precise identification of the specific amino acids involved in G protein activation and lead to a molecular understanding of the activation mechanism as cognate amino acids identified on individual GPCRs. Some of the specific aims of the present invention are described below.

Replacement of 35 C terminal amino acids of Gi1α with those from Gqα was not sufficient to permit coupling of the chimeric Gα subunit to M1 receptors. Addition of the 6 N-terminal amino acids unique to Gqα was also not sufficient to permit coupling. However, according to the present invention, chimeric Gα subunits containing both Gq N- and C-terminal sequences in a Gi1α context did functionally interact with M1 receptors.

There are four major families of G proteins and in general, receptors interact functionally with just one family of G proteins. Members of the Gi/o family of G proteins are easily expressed and purified because of their inherent solubility. Members of the Gq family are difficult to purify in functional form, in part because of their low solubility. The present invention solved the solubility problem of the Gq family by contructing chimeric α subunits by using a Gq α subunit as the donor alpha and a Gi/o α subunit as the recipient alpha. The resulting chimeric α subunit is soluble in water and has receptor coupling selectivity of G proteins having Gq α subunits. The soluble chimeric G protein could be used in a variety of assays investigating properties of Gq-coupled receptors, especially in drug-discovery assays. An example of the soluble chimeric G protein is a G protein having Gαi1q6N35C as a chimeric α subunit, a β subunit (such as β1) and a γ subunit (such as γ1 or γ2).

With the idea of replacing or inserting appropriate N-terminal and C-terminal amino acids in the α subunit of a G protein disclosed above in mind, one skilled in the art can make the chimeric α subunit using molecular genetic, biochemical or chemical techniques known in the art. As an illustration, some of the chimeric α subunits can be made by sequence manipulations described below.

Construction of Gi1q6N

The rat Gi1α sequence was used to construct pVLSGGi1 for expression of the native protein in the baculovirus expression system (BES). This construct was described in Graber et. al, J. Biol. Chem. 267:1271-1278 (1992). The nucleotides coding for the 6 amino terminal acids of mouse Gqα were inserted into the BamH1/Nco1 sites of pVLSGGi1 using synthetic oligonucleotides to create the duplex linker shown below. The rat Gi1α has an Nco1 site at its ATG start codon such that the 6 Gqα N-terminal amino acids are added in frame by this strategy. The strategy was complicated by an internal Nco1 site in the Gi1α sequence, however partial digestion of pVLSGGi1 with Nco1 and sequencing of the final construct produced the anticipated pVLSG-Gi1Q6N. This in turn has been used to create a recombinant baculovirus producing the chimeric G protein known as Gi1Q6N. A silent mutation (CTCGAG, SEQ ID NO:19, instead of CTGGAG, SEQ ID NO:20) in the linker sequence introduced a unique XhoI site (underlined) without changing the amino acid sequence of native mouse Gqα (MTLESI). The initiating ATG codon is also underlined.

| | |
|---|---|
| 5′-GATCCATGACTCTCGAGTCCAT | (SEQ ID NO:21) |
| GTACTGAGAGCTCAGGTAGTAC-3′ | |

Construction of Gi1q6N3C

Gi1q6N3C was constructed first and used as the parent for the production of Gi1q6N11C and Gi1q6N35C. Each of these were in turn constructed from Gi1q3C, Gi1q11C and Gi1q35C which were made by Hyunsu Bae in Heidi Hamm's laboratory. The preparation of Bae's constructs is described here. H₆pQE-60-Gi1 was used as a parent for C-terminal replacements. H₆pQE-60-Gi1 is an expression plasmid for rat Gi1α and was generated by Maurine Linder as described in Methods in Enzymology 237:146-164 (1994). Notably, it lacks the internal Nco1 site at bp 260 of Gi1α coding sequence. Bae's C-terminal replacements were made using PCR and verified by sequencing to have the 3, 11 or 35 C-terminal amino acids of Gi1α replaced by those from Gqα (there are no gaps in the sequence alignment of Gi1α and Gqα over the C-terminal 35 amino acids). Bae also introduced a unique BamH1 site in the vicinity of amino acid 214 of Gi1α without changing the coding sequence. Therefore the C-terminal portion of these chimeras could be excised using the BamH1 site and a 3′ HindIII site in the H₆pQE-60 vector. To add the 6 N-terminal amino acids from Gqα to Bae's Gi1q3C construct, Bae's H₆pQE-60-Gi1q3C was digested with EcoR1 and Nco1. EcoR1 cuts vector sequence 5′ of the ATG start codon for Gi1α and Nco1 cuts at the start codon. Synthetic oligonucleotides were then used to create the duplex linker shown below to religate Bae's linearized plasmid resulting in H₆pQE-60-Gi1q6N3C. The duplex linker creates an Nco1 site at the N-terminal methionine of Gqα while abolishing the Nco1 site at the Gi1α ATG and places the 6 N-terminal amino acids of Gqα in-frame with those of Gila. The initiating ATG for the N-terminal methionine from Gqα is underlined.

| | |
|---|---|
| 5′-AATTCCATGGATGACTCTCGAGTCCAT | (SEQ ID NO:22) |
| GGTACCTACTGAGAGCTCAGGTAGTAC-3′ | |

To express the protein in the BES and take advantage of the unique BamH1 site described above to create Gi1Q6N11C and Gi1Q6N35C, the BamH1 site was removed and an Nco1 site was added to the commercially available baculovirus expression vector pVL1393 by inserting the duplex linker shown below between the BamH1 and XbaI sites of the pVL1393 poly-linker. The modified pVL1393 has been designated pVLKD

| | |
|---|---|
| 5′-GATCTCCATGGCCCGGGT | (SEQ ID NO:23) |
| AGGTACCGGGCCCAGATC-3′ | |

To subclone the Gi1q6N3C coding sequence into pVLKD the NcoI/HindIII fragment of H₆pQE-60-Gi1q6N3C was inserted in the Nco1/EcoRI sites of pVLKD using the duplex linker shown below:

| | |
|---|---|
| 5′-AGCTGTATCTAGATAG | (SEQ ID NO:24) |
| CATAGATCTATCTTAA-3′ | |

This created plasmid pVLKD-Gi1q6N3C which was used to create a baculovirus expressing the chimeric Gi1q6N3C protein, and as the parent for the construction of pVLKD-Gi1q6N11C and pVLKD-Gi1q6N35C described below.

Construction of Gi1q6N11C and Gi1q6N35C

The BamH1/HindIII fragment of Bae's H₆pQE-60-Gi1q11C and H₆pQE-60-Gi1q35C were subcloned into the BamH1/EcoRI sites of pVLKD-Gi1q6N3C using the duplex linker described above for the construction of pVLKD-Gi1q6N3C. This resulted in the replacement of the BamH1/HindIII fragment of pVLKD-Gi1q6N3C with those from H₆pQE-60-Gi1q11C and H₆pQE-60-Gi1q35C creating pVLKD-Gi1q6N11C and pVLKD-Gi1q6N35C.

The sequences of all constructs used to produce baculoviruses have been verified by DNA sequencing. Baculoviruses expressing Gi1q6N, Gi1q6N3C, Gi1q6N11C and Gi1q6N35C have been isolated. With the exception of Gi1q6N11C, each of these proteins has been purified and tested for interaction with M1 and M2 muscarinic receptors. Data not shown indicate that Gi1q6N3C did not functionally interact with either receptor.

A poorly defined region (residues 1-219) has been identified in the N-terminal half of ail as contributing to optimal functional interactions with both 5-HT_{1A} and 5-HT_{1B} receptors. The unique six amino acid extension at the N-terminus of Gq family members when added to the amino terminus of ail has been shown to be insufficient to prevent coupling of the chimera to 5-HT₁ receptors. The role of the extreme C-terminus of αi1 in 5-HT₁ receptor coupling has been studied using chimeric αi1 subunits in which 3, 5, 11 or 35 C-terminal amino acids have been replaced with those from αq. Coupling to 5-HT_{1B} receptors was eliminated by replacement of just the three C-terminal amino acids of ail, and although coupling to 5-HT_{1A} receptors was impaired with Giq3C and Giq5C, it was not eliminated until eleven C-terminal amino acids of αi1 were replaced with those from αq. These data are shown in FIG. 7 and discussed more completely below.

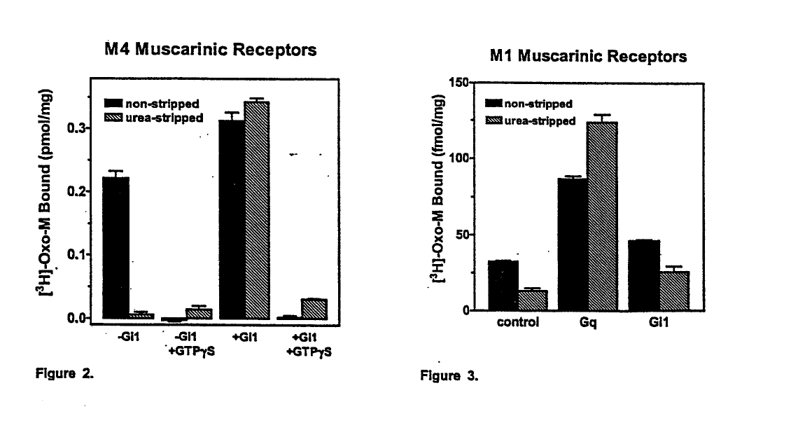

Successful expression and coupling of M2 muscarinic receptors was obtained. Coupling of M1, M4 and M5 receptors has been more difficult to establish, primarily because of unexpectedly low expression of the M4 receptor and apparent nearly complete coupling of M1 and M5 receptors with endogenous Sf9 cell G proteins, even when the receptors were expressed at considerable levels (>4 pmol/mg membrane protein). As shown for M4 and M1 receptors in FIGS. 2 and 3, urea stripping Sf9 cell membranes containing the expressed receptors [80] has effectively overcome these limitations. The apparently greater efficiency of this protocol for M4 receptors is likely due to using two urea treatments for the M4 receptors compared with one for the M1 receptors. Due to much higher expression levels of the M2 receptor (often >20 pmol/mg), urea stripping of membranes is not required for effective coupling. Control experiments (data not shown) have demonstrated that comparable results were obtained with non-stripped and urea-stripped M2 expressing membranes, though for reasons explained above, the magnitude of the affinity shift was significantly greater in the stripped membranes. Despite numerous attempts, in both non-stripped and urea-stripped membranes, the M3 muscarinic receptor has not been successfully been coupled with any exogenous G proteins. It appears that additional components, not present in Sf9 cell membranes, are required for G protein coupling of the M3 receptor.

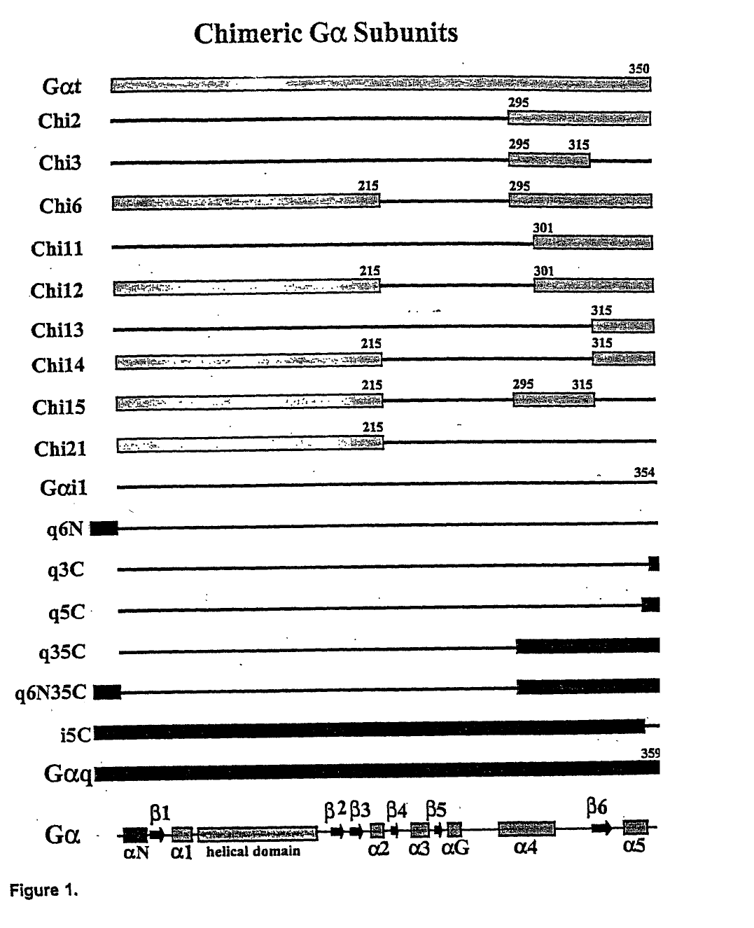

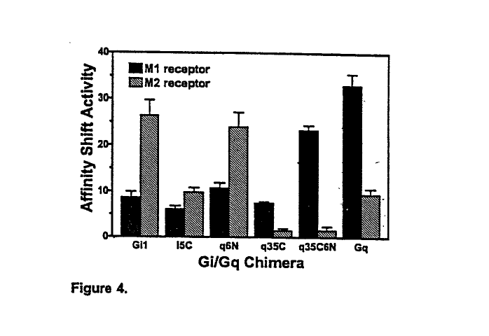

Substantial progress has been made in identifying the domains responsible for the general coupling selectivity of M1 receptors for Gq and M2 receptors for Gi1. FIG. 1 presents the secondary structures of all G chimeras discussed herein which have been expressed and purified from either bacteria [44,51] or Sf9 cells [78]. Previous work of Bourne, Conklin, Wess and others (discussed above) [33-37] demonstrated that the extreme C-terminus of Gi1α when placed in the context of Gqα is sufficient to permit Gi-coupled receptors to stimulate PLC-β via chimeric Gq/Gi proteins. The importance of this region was confirmed by demonstrating that the extreme C terminus of Gqα when placed in the context of Gi1α prevents coupling of chimeric Gi/Gq subunits to muscarinic M2 receptors. (FIGS. 4 & 7) However, even 35 C-terminal amino acids of Gqα when placed in the context of Gi1α are not sufficient to permit coupling of the chimeric Gi/Gq subunits to M1 muscarinic receptors (FIG. 4). Furthermore, purification of the Gqi5 chimera used in the transfection studies of Bourne, Conklin and Wess (cDNA kindly provided by Bruce Conklin) after expression in Sf9 cells and reconstitution as a heterotrimer with M1 and M2 receptors demonstrates that just 5 C-terminal amino acids of Gi1α placed in the context of Gqα prevents M1 receptor coupling while allowing only minimal coupling with M2 receptors. (FIG. 4) The unique 6 amino acid N-terminal extension of Gqα has been added to the N-terminus of Gi1α and the chimera has been expressed and purified from Sf9 cells. In the context of a heterotrimer this chimeric α subunit neither prevented M2 receptor coupling, as surmised from transfection studies [46], nor permitted M1 receptor coupling (FIG. 4). However, the q6N35C chimera, which is substantially Gi1 in character, gained the ability to productively couple M1 receptors and lost the ability to couple M2 receptors. (FIG. 4). Interestingly, the activity of the q6N35C chimera implies that the α4-helix and α4-β6 loop region is not likely to be a selectivity determinant for M1 receptors despite it's critical role in coupling M2 and several serotonin receptors .

The data in FIG. 4, taken together, indicate that different regions of Gi1α and Gqα are responsible for their selective interactions with muscarinic M1 and M2 receptors, and that receptor coupling in reconstitution studies differs from that in transfection studies. The coupling in transfection may be more efficient than in reconstitution, other signaling events may contribute to effector (phospholipase C in the studies cited) activation, or the amplification at the receptor-G protein and G protein-effector interfaces may combine to produce nearly maximal effector stimulation with sub-optimal G protein coupling. Regardless, the reconstitution clearly adds additional information regarding the relative importance of distinct regions not implicated in the transfection studies and is sensitive enough to allow identification of the individual amino acids within these regions.

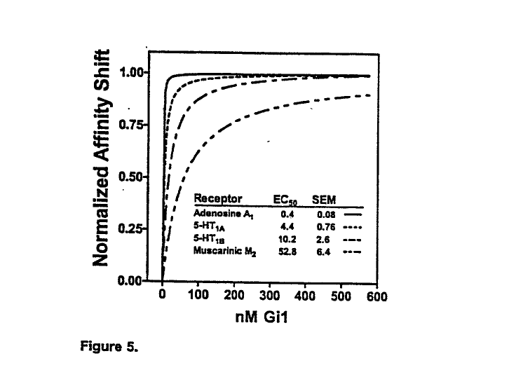

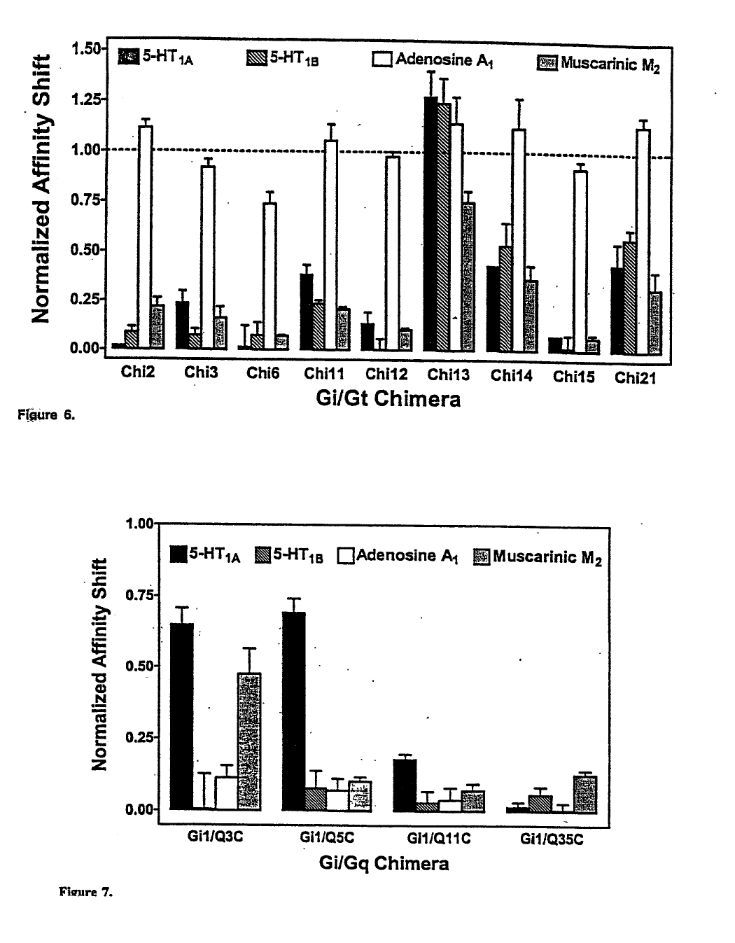

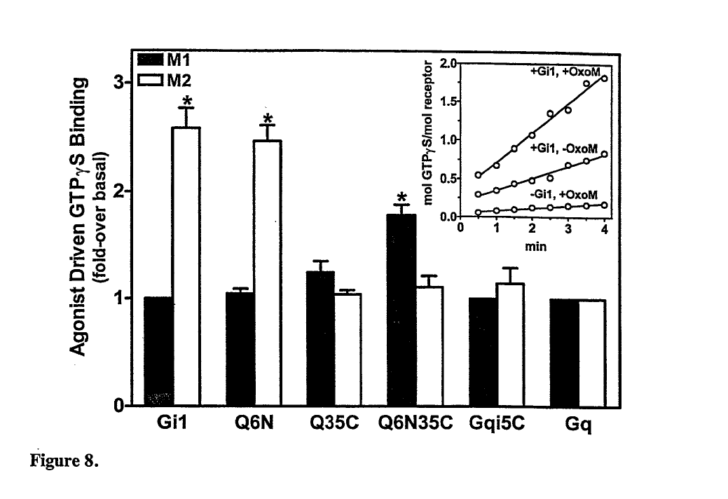

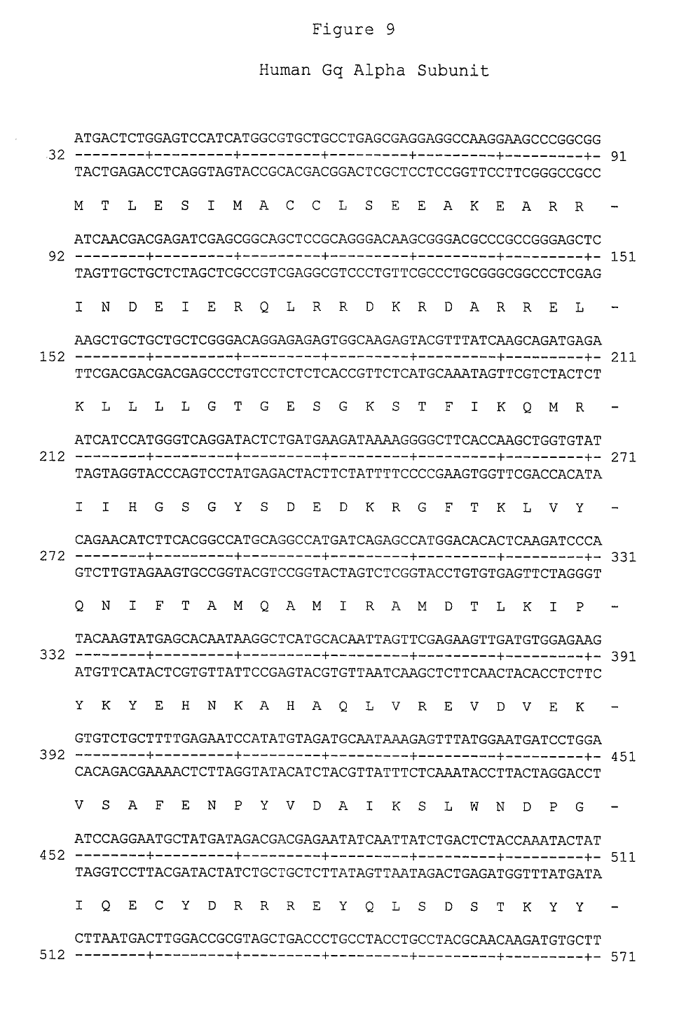

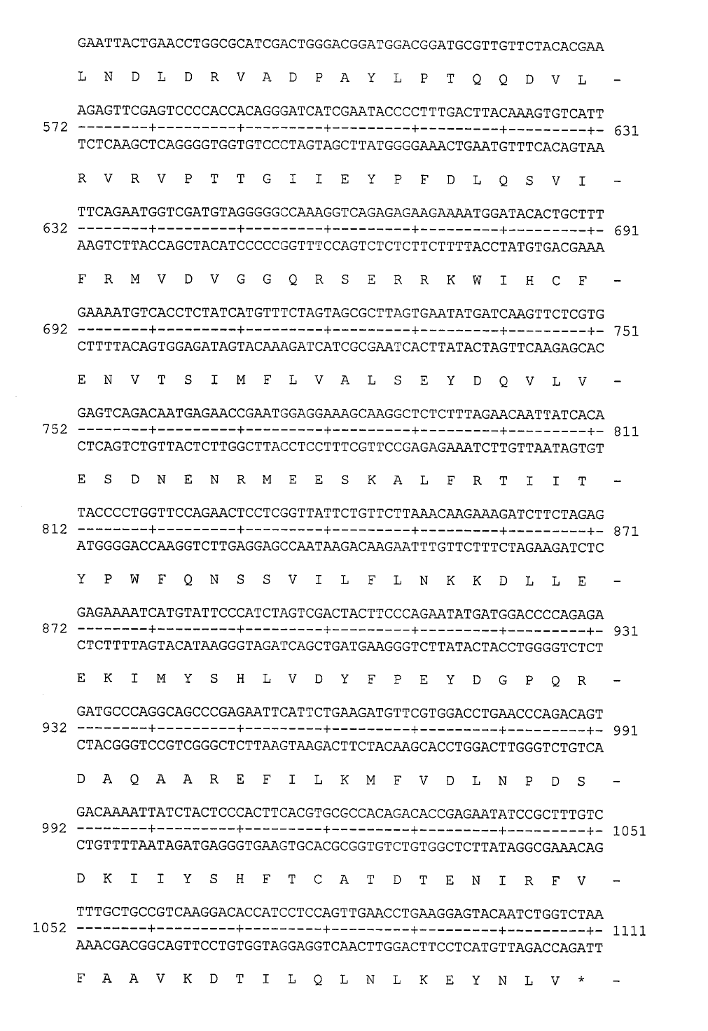

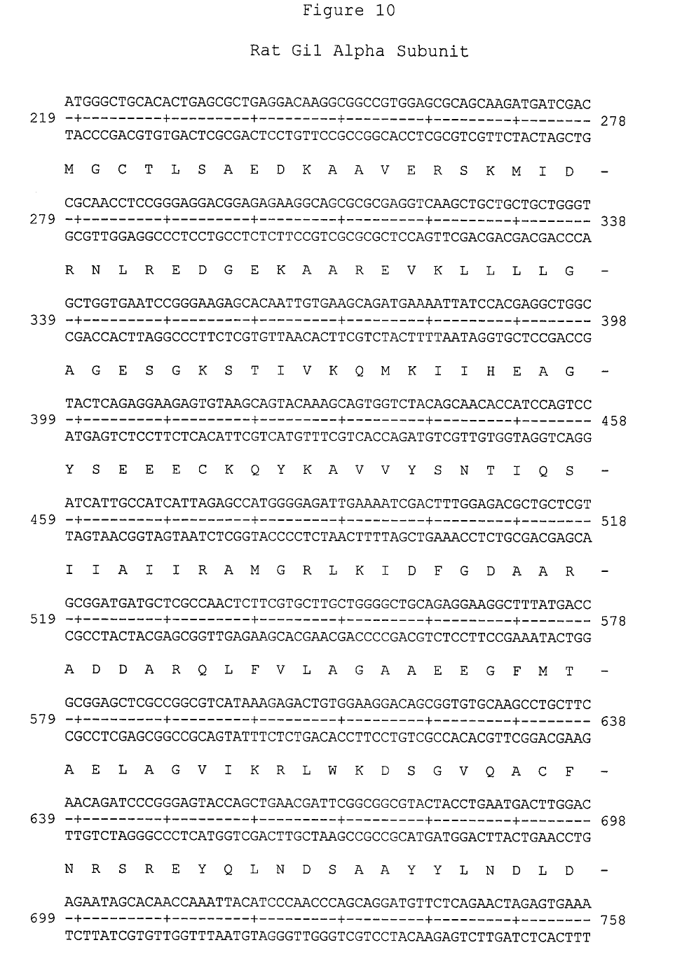

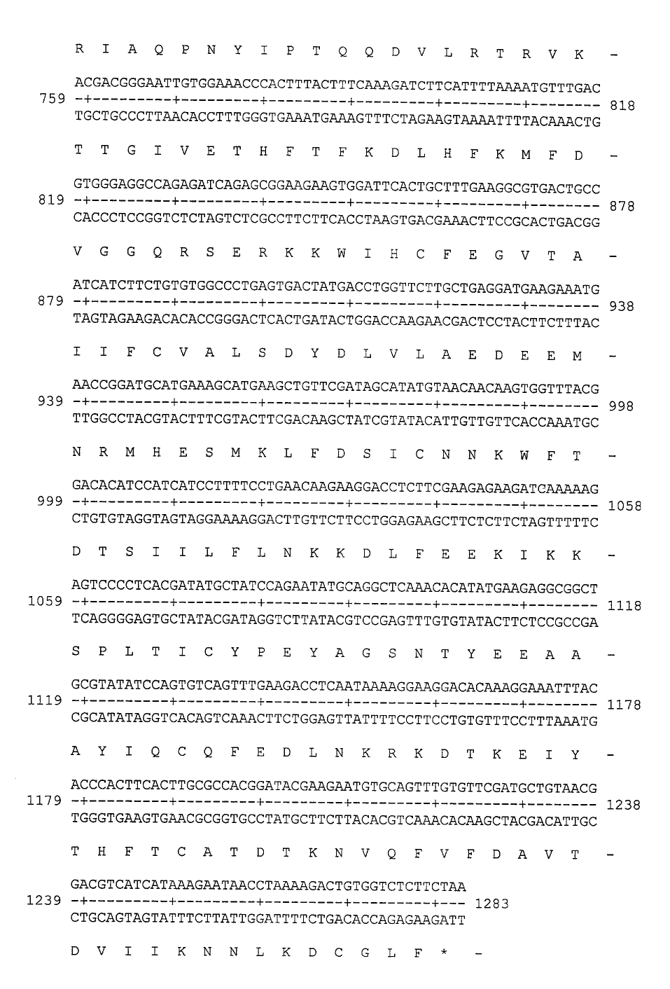

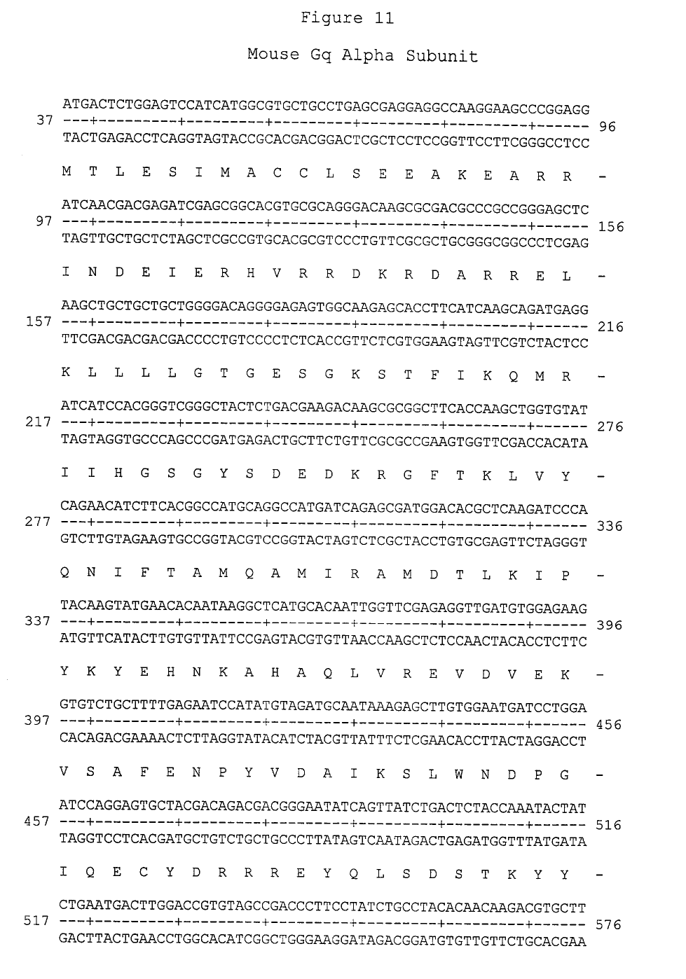

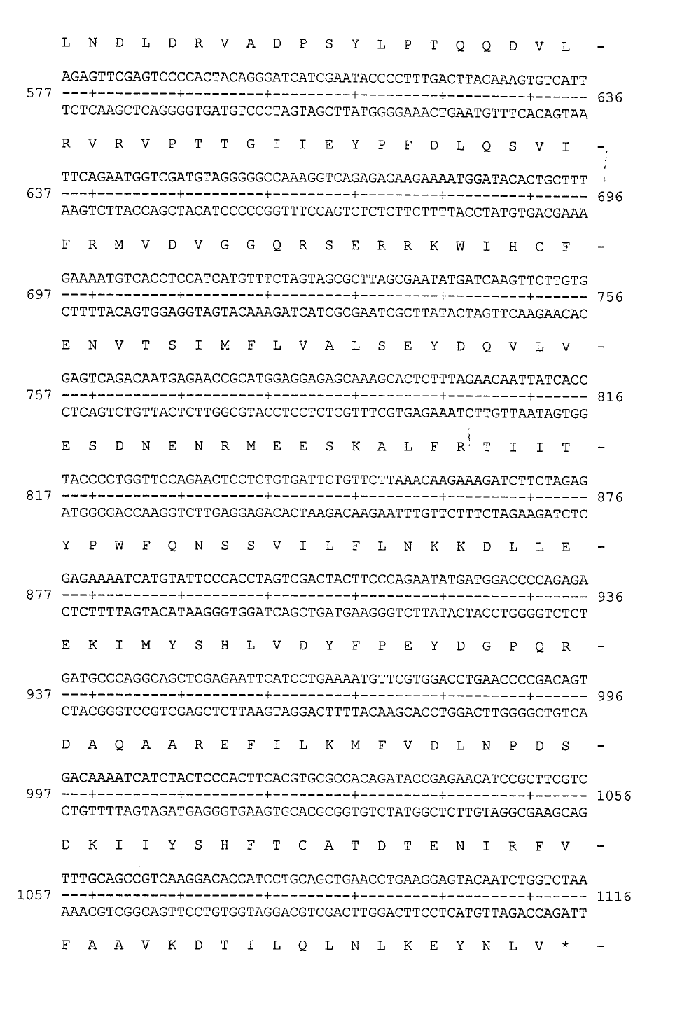

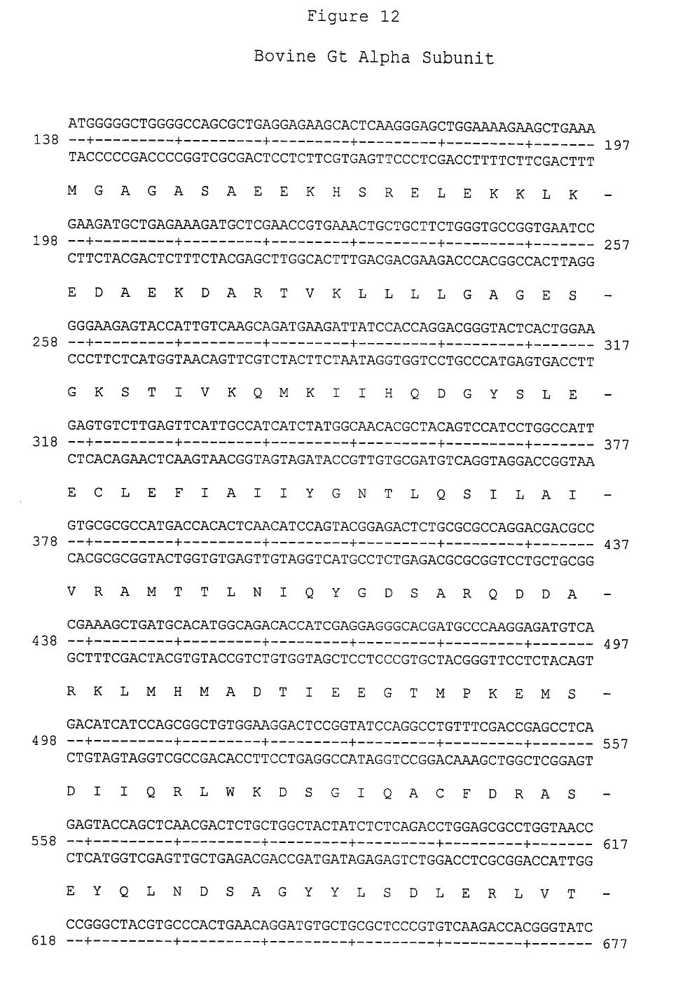

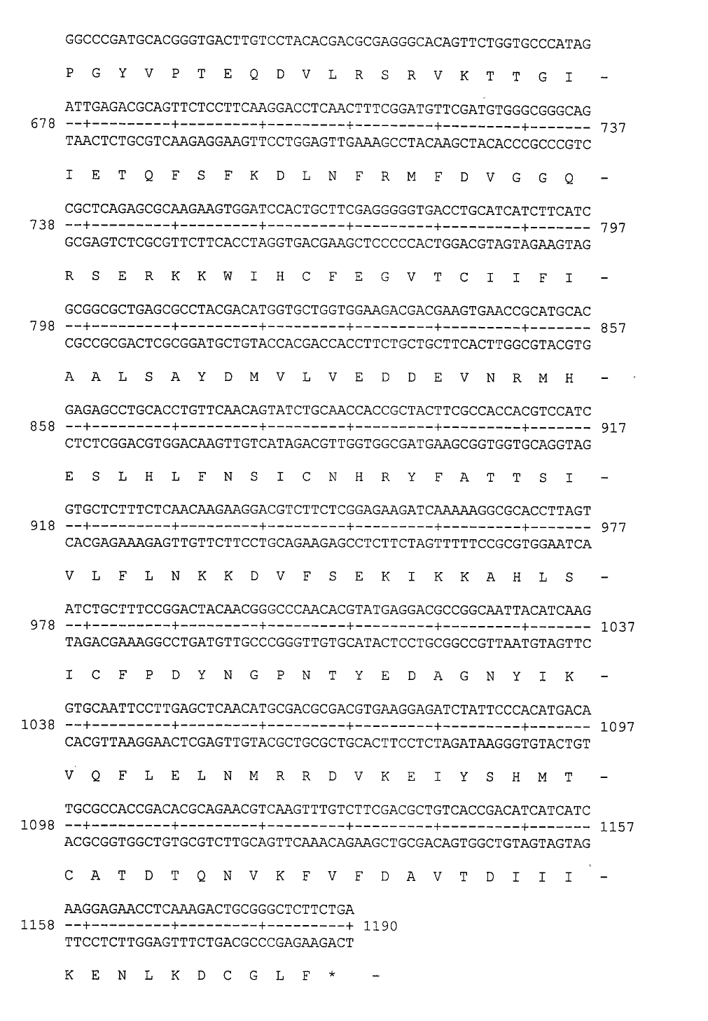

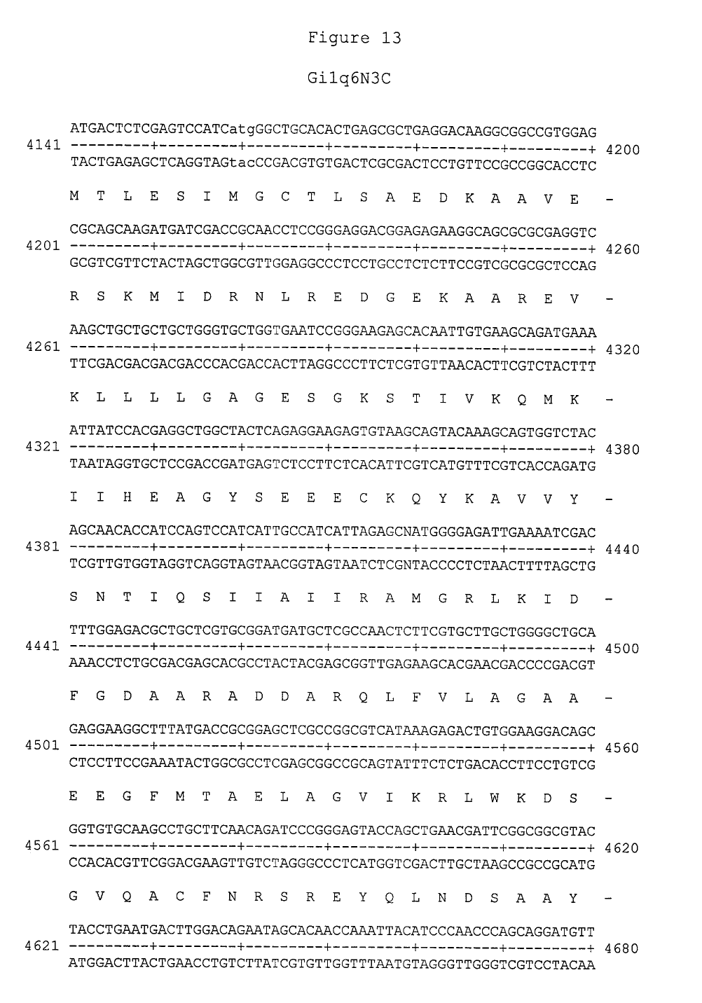

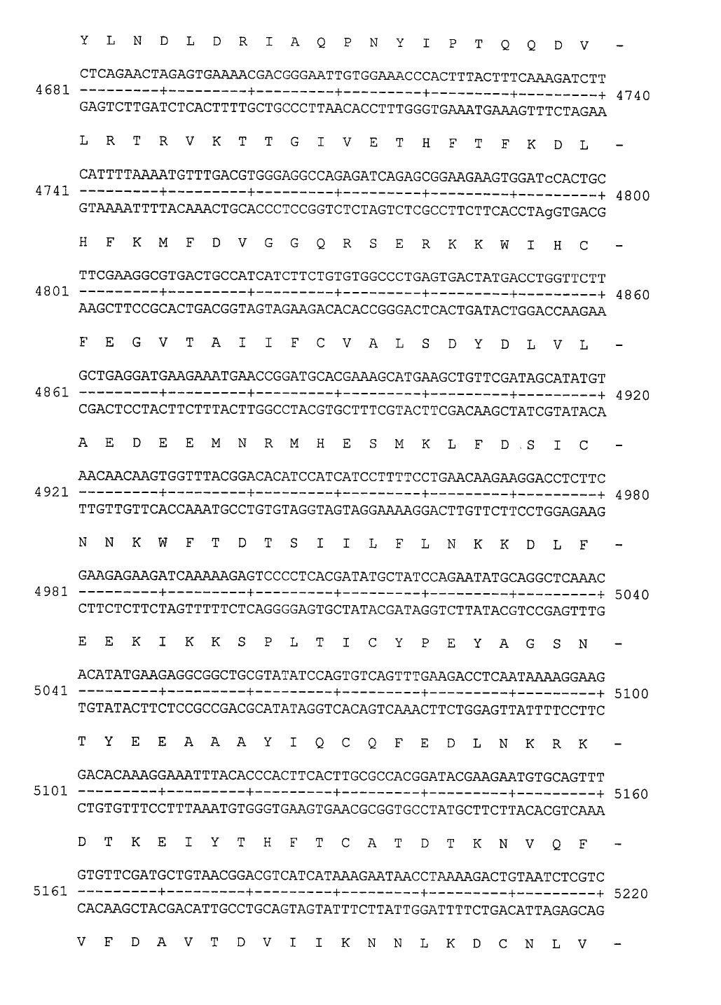

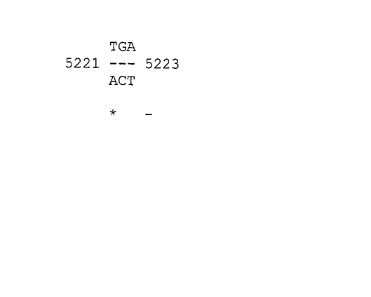

The inventor's data show that even the closely related serotonin 5HT_{1A} and 5-HT_{1B}, A₁, adenosine and M2 muscarinic receptors differ significantly in the basic parameters underlying functional interactions with their cognate G proteins. As shown in FIG. 5, establishing the G protein dependence of affinity shift activity for these receptors revealed that the apparent affinity with which they interact with Gi1 differs significantly. While all four receptors are members of Family A (Rhodopsin-like) of the GPCR superfamily, the 5HT_{1A}, 5-HT_{1B} and M2 muscarinic are biogenic amine receptors while the A₁ adenosine receptor is more closely related to the cannabinoid, melanocortin and olfactory receptors [84]. As shown in FIG. 6, the A₁ adenosine receptor does not distinguish between Gail and Gat sequences, while the serotonin 5-HT_{1A} and 5-HT_{1B} and muscarinic M2 receptors require Gai1 residues within the α4 helix-α4/β6 loop region (amino acids 299-318) and N-terminus for optimal coupling. In collaboration with Heidi Hamm, the inventor has demonstrated that Q304 and E308 in the α4 helix of αil are primarily responsible for this discrimination by 5-HT1B receptors [51]. Using both loss of function and gain of function criteria these same two amino acids have been shown to be critical for the αi1/αt discrimination exhibited by the other receptors as well (data not shown). All of the receptors examined are sensitive to replacement of C terminal Gi1α residues with those from Gαq, confirming the importance of this domain for coupling [33-37]. However, the receptors differ in their sensitivity to replacements in the C terminus. As shown in FIG. 7, replacement of 3 Gi1α C-terminal residues with those from Gqα eliminates coupling with 5-HT_{1B} and A₁ adenosine receptors, while replacements of 5 and 11 C-terminal residues are required to eliminate coupling to muscarinic M2 and 5-HT_{1A} receptors respectively. As before [44,51], the results from affinity shift assays correlate strongly with results from agonist driven GTPγS binding assays (data not shown). Thus the biogenic amine receptors seem to require appropriate sequences at the N- and C-termini as well as within the α4 helix for optimal coupling with Gα subunits. The related A₁ adenosine receptor does not distinguish between αi1 and αt sequences within the N-terminus and a4 helix (αi1 and at have identical 5 C- terminal amino acids), but does use the extreme C- terminus as a critical domain for receptor selectivity.

Some aspects of the present invention focus on muscarinic receptors. Muscarinic receptors exhibit ratios as high as 30,000-fold for the high and low affinity states for agonists such as oxotremorine M (Oxo-M) and acetylcholine [85,86]. Such a difference in affinities has been exploited for a far more sensitive affinity shift assay than that afforded by the roughly 40-200 fold differences in high and low agonist affinities of most other GPCRs in Sf9 cell membranes [78,87]. Affinity shift activities from 50-250 are regularly achieved with urea stripped membranes (see FIG. 2 for example). Thus the affinity shift assay has the sensitivity to distinguish roles of individual amino acids in the selectivity for αi1, αt and αq subunits exhibited by the five muscarinic subtypes. The low non-specific binding and good stability exhibited by the commercially available radioligands Oxotremorine-M and N-methyl scopolamine allow convenient and relatively inexpensive binding assays. Most importantly, the wealth of information regarding the functional roles of individual amino acids in muscarinic receptors will nicely complement the identification of individual amino acids within Gα subunits and should ultimately lead to understanding of the activation process at the molecular level. While the rhodopsin-transducin interface is likely to be the first completely mapped it will nevertheless be important to understand other G protein-receptor interfaces.

All the methods required for the preparation of the chimeric α subunits of the present invention are either known in the art or have been described in the present application. The construction of the proposed chimeric α subunits can be facilitated by the ability to swap domains among existing chimeras as useful restriction sites have been engineered into many of these constructs. However use of “RNA- and DNA- overhang cloning” can eliminate the need for restriction enzymes in gene engineering and allows the recombination of DNA fragments at any location without the insertion, deletion or alteration of even a single base pair if so desired [88]. Thus chimeras of interest can be generated solely from structural considerations regardless of the existence of convenient restriction sites.

### REFERENCES CITED

1. Hildebrandt J D: “Role of subunit diversity in signaling by heterotrimeric G proteins” Biochem.Pharmacol. 54, 325-339, 1997

2. Hebert T E and Bouvier M: “Structural and functional aspects of G protein-coupled receptor oligomerization” Biochem. Cell Biol. 76, 1-11, 1998

3. Premont R T, Inglese J, and Lefkowitz R J: “Protein kinases that phosphorylate activated G protein-coupled receptors.” FASEB J. 9, 175-182, 1995

4. Daaka Y, Pitcher J A, Richardson M, Stoffel R H, Robishaw J D, and Lefkowitz R J: “Receptor and Gpy isoform-specific interactions with G protein-coupled receptor kinases.” Proc.Natl.Acad.Sci.U.S.A. 94, 2180-2185, 1997

5. Pronin A N and Benovic J L: “Regulation of the G protein-coupled receptor kinase GRK5byproteinkinaseC.” J. Biol. Chem. 272, 3806-3812, 1997

6. Daaka Y, Luttrell L M, Ahn S, Della Rocca G J, Ferguson S S, Caron M G, and Lefkowitz R J: “Essential role for G protein-coupled receptor endocytosis in the activation of mitogen-activated protein kinase” J. Biol . Chem. 273, 685-688, 1998

7. Lefkowitz R J: “G protein-coupled receptors. III. New roles for receptor kinases and beta-arrestins in receptor signaling and desensitization” J. Biol. Chem. 273, 18677-18680, 1998

8. Luttrell L M, Hawes B E, van Biesen T, Luttrell D K, Lansing T J, and Lefkowitz R J: “Role of c-Src tyrosine kinase in G protein-coupled receptor- and Gpy subunit-mediated activation of mitogen-activated protein kinases” J. Biol. Chem. 271, 19443-19450, 1996

9. Luttrell L M, Ferguson S S, Daaka Y, Miller W E, Maudsley S, Della Rocca G I, Lin F, Kawakatsu H, Owada K, Luttrell D K, Caron M G, and Lefkowitz R J: “Beta-arrestin-dependent formation of (2 adrenergic receptor-Src protein kinase complexes [see comments]” Science 283, 655-661, 1999

10. McLatchie L M, Fraser N J, Main M J, Wise A, Brown I; Thompson N, Solari R, Lee M G, and Foord S M: “RAMPs regulate the transport and ligand specificity of the calcitonin-receptor-like receptor” Nature 393, 333-339, 1998

11. Dohlman H G and Thorner J: “RGS proteins and signaling by heterotrimeric G proteins.” J. Biol. Chem. 272, 3871-3874, 1997

12. De Vries L and Gist F M: “RGS proteins: more than just GAPs for heterotrimeric G proteins” Trends Cell Biol. 9, 138-144, 1999

13. Huang C, Hepler J R, Gilman A G, and Mumby S M: “Attenuation of Gi- and Gq-mediated signaling by expression of RGS4 or GAIP in mammalian cells.” Proc.Natl.Acad.Sci.U.S.A. 94, 6159-6163, 1997

14. Heximer S P, Watson N, Linder M E, Blumer K J, and Hepler J R: “RGS2/GOS8 is a selective inhibitor of Gqalpha function.” Proc.Natl.Acad. Sci.USA 94, 14389-14393, 1997

15. Xu X, Zeng W, Popov S, Berman D M, Davignon I, Yu K, Yowe D, Offermanns S, Muallem S, and Wilkie T M: “RGS proteins determine signaling specificity of Gq-coupled receptors” J. Biol. Chem. 274, 3549-3556, 1999

16. Cismowski M J, Takesono A, Ma C, Lizano J S, Xie X, Fuernkranz H, Lanier S M, and Duzic E: “Genetic screens yeast to identify mammalian nonreceptor modulators of G-protein signaling” Nat .Biotechnol. 17, 878-883, 1999

17. Takesono A, Cismowski M J, Ribas C, Bernard M, Chung P, Hazard S, III, Duzic E, and Lanier S M: “Receptor-independent activators of heterotrimeric G-protein signaling pathways” J. Biol. Chem. 274, 33202-33205, 1999

18. Natochin M, Lester B, Peterson Y K, Bernard M L, Lanier S M, and Artemyev NO: “AGS3 Inhibits GDP association from Ga1phα subunits of the Gi Family and Rhodopsin-dependent Activation of Transducin” J. Biol. Chem. 275, 40981-40985, 2000

19. Peterson Y K, Bernard ML, Ma H, Hazard S, III, Graber S G, and Lanier S M: “Stabilization of the GDP-bound conformation of gialpha by a peptide derived from the G-protein regulatory motif of AGS3” J. Biol. Chem. 275, 33193-33196, 2000

20. Bernard M L, Peterson Y K, Chung P, Jourdan J, and Lanier S M: “Selective interaction of AGS3 with G-proteins and the influence of AGS3 on the activation stateofG-proteins” J. Biol. Chem. 276, 1585-1593, 2001

21. Cismowski M J, Ma C, Ribas C, Xie X, Spruyt M, Lizano J S, Lanier S M, and Duzic E: “Activation of heterotrimeric G-protein signaling by a ras-related protein. Implications for signal integration” J. Biol. Chem. 275, 23421-23424, 2000

22. Noel J P, Hamm H E, and Sigler P B: “The 2.2 Angstrom crystal structure of transducin-a complexed with GTP\(*gS” Nature 366, 654-663, 1993

23. Lambright D G, Noel J P, Hamm H E, and Sigler P B: “Structural determinants for activation of the a-subunit of a heterotrimeric G protein”. Nature 369, 621-628, 1994

24. Mixon M B, Lee E, Coleman D E, Berghuis A M, Gilman A G, and Sprang S R: “Tertiary and quaternary structural changes in G₁ₐ₁ induced by GTP hydrolysis” Science 270, 954-960, 1995

25. Sunahara R K, Tesmer J J, Gilman A G, and Sprang S R: “Crystal structure of the adenylyl cyclase activator Gsalpha” Science 278, 1943-1947, 1997

26. Sondek J, Bohm A, Lambright D G, Hamm H E, and Sigler P B: “Crystal structure of a GA protein b gamma diner at 2.1A resolution” Nature 379, 369-374, 1996

27. Lambright D G, Sondek J, Bohm A, Skiba N P, Hamm H E, and Sigler PB: “The 2.0 A crystal structure of a heterotrimeric G protein” Nature 379, 311-319, 1996

28. Wall M A, Coleman D E, Lee E, Iniguez-Lluhi J A, Posner B A, Gilman A G, and Sprang S R: “The structure of the G protein heterotrimer Gia1b1g2” Cell 83, 1047-1058, 1995

29. Dratz E A, Furstenau J E, Lambert C G, Thireault D L, Rarick H, Schepers T, Pakhlevaniants S, and Hamm H E: “NMR structure of a receptor-bound G-protein peptide” Nature 363, 276-281, 1993

30. Martin E L, Rens-Domiano S, Schatz P J, and Hamm HE: “Potent peptide analogues of a G protein receptor-binding region obtained with a combinatorial library” J. Biol. Chem. 271, 361-366, 1996

31. Garcia P D, Onrust R, Bell S M, Sakmar T P, and Bourne H R: “Transducin-a C-terminal mutations prevent activation by rhodopsin: a new assay using recombinant proteins expressed in cultured cells” EMBO J. 14, 4460-4469, 1995

32. Rasenick M M, Watanabe M, Lazarevic M B, Hatta S, and Hamm H E: “Synthetic peptides as probes for G protein function. Carboxyl- terminal Gₐₛ pept ides mimic Gₛ and evoke high affinity agonist binding to b-adrenergic receptors” J. Biol. Chem. 269, 21519-21525, 1994

33. Conklin B R, Farfel Z, Lustig K D, Julius D, and Bourne H R: “Substitution of three amino acids switches receptor specificity of G_{qa} to that of G_{i a}” Nature 363, 274-276, 1993

34. Liu J, Conklin B R, Blin N, Yun J, and Wess J: “Identification of a receptor/G-protein contact site critical for signaling specificity and G-protein activation” Proc.Natl.Acad.Sci.USA. 92, 11642-11646, 1995

35. Conklin B R, Herzmark P, Ishida S, Voyno-Yasenetskaya T A, Sun Y, Farfel Z, and Bourne H R: “Carboxyl-terminal mutations of Gq alpha and Gs alpha that alter the fidelity of receptor activation.” Mol.Pharmacol. 50, 885-890, 1996

36. Kostenis E, Conklin B R, and Wess J: “Molecular basis of receptor/G protein coupling selectivity studied by coexpression of wild type and mutant m2 muscarinic receptors with mutant Gαq subunits.” Biochemistry 36, 1487-1495, 1997

37. Kostenis E, Gomeza J, Lerche C, and Wess J: “Genetic analysis of receptor-Galphaq coupling selectivity.” J. Biol. Chem. 272, 23675-23681, 1997

38. Slessareva, J. S. and Graber, S. G.: “Molecular Determinants of Selectivity in M1 and M2 Muscarinic Receptor Coupling with Gq and Gi1 Subunits” FASEB Abstr. 14, A1487 2000

39. Ma, H., Depree, K. M., Cabrera-Vera, T. M., Slessareva, J. E., Bae, H, Hamm, H. E., and Graber, S. G.: “Domains of G Protein α subunits Involved in Receptor Coupling” FASEB Abstr 14, A1342 2000

40. Natochin M, Muradov K G, McEntaffer R L, and Artemyev N O: “Rhodopsin recognition by mutant G(s)alpha containing C-terminal residues of transducin” J. Biol. Chem. 275, 2669-2675, 2000

41. Hamm H E, Deretic D, Arendt A, Hargrave P A, Koenig B, and Hofmann K P: “Site of G protein binding to rhodopsin mapped with synthetic peptides from the a subunit” Science 241, 832-835, 1988

42. Higashijima T and Ross EM: “Mapping of the mastoparan-binding site on G proteins. Cross- linking of [¹²⁵I-Tyr³, Cys¹¹] mastoparan to Gₒ” J. Biol. Chem. 266, 12655-12661, 1991

43. Taylor J M, Jacob-Mosier G G, Lawton R G, Remmers A E, and Neubig R R: “Binding of an alpha 2 adrenergic receptor third intracellular loop peptide to G beta and the amino terminus of G alpha” J. Biol. Chem. 269, 27618-27624, 1994

44. Bae H, Anderson K, Flood L A, Skiba N P, Hamm H E, and Graber S G: “Molecular Determinants of Selectivity in 5-Hydroxytrytamine1B Receptor-G Protein Interactions.” J. Biol. Chem. 272, 32071-32077, 1997

45. Hepler J R, Biddlecome G H, Kleuss C, Camp L A, Hofmann S L, Ross E M, and Gilman A G: “Functional importance of the amino terminus of Gq alpha” J. Biol. Chem. 271, 496-504, 1996

46. Kostenis E, Degtyarev M Y, Conklin B R, and Wess J: “The N-terminal extension of Gaphaq is critical for constraining the selectivity of receptor coupling.” J.Biol.Chem. 272, 19107-19110, 1997

47. Lee C H, Katz A, and Simon M I: “Multiple regions of Gal6 contribute to the specificity of activation by the C5a receptor” Mol. Pharmacol. 47, 218-223, 1995

48. Hamm H E, Deretic D, Arendt A, Hargrave P A, Koenig B, and Hofmann K P:

“Site of G protein binding to rhodopsin mapped with synthetic peptides from the alphα subunit” Science 241, 832-835, 1988

49. Onrust R, Herzmark P, Chi P, Garcia P D, Lichtarge O. Kingsley C, and Bourne HR: “Receptor and betagamma binding sites in the alphα subunit of the retinal G proteintransducin” Science 275, 381-384, 1997

50. Natochin M, Granovsky A E, Muradov K G, and Artemyev N O: “Roles of the transducin alpha-subunit alpha4-helix/alpha4-beta6 loop in the receptor and effector interactions” J. Biol. Chem. 274, 7865-7869, 1999

51. Bae H, Cabrera-Vera T M, Depree K M, Graber S G, and Hamm H E: “Two amino acids within the alpha4 helix of Galphail mediate coupling with 5-hydroxytryptamine1B receptors” J. Biol . Chem. 274, 14963-14971, 1999

52. Fung B K: “Characterization of transducin from bovine retinal rod outer segments. I. Separation and reconstitution of the subunits” J. Biol. Chem. 258, 10495-10502, 1983

53. Florio V A and Sternweis P C: “Reconstitution of resolved muscarinic cholinergic receptors with purified GTP-binding proteins” J. Biol . Chem. 260, 3477-3483, 1985

54. Kelleher D J and Johnson G L: “Transducin inhibition of light-dependent rhodopsin phosphorylation: evidence for beta gammα subunit interaction with rhodopsin” Mol.Pharmacol. 34, 452-460, 1988

55. Phillips W J and Cerione R A: “Rhodopsin/transducin interactions. I. Characterization of the binding of the transducin-beta gammα subunit complex to rhodopsin using fluorescence spectroscopy” J. Biol . Chem. 267, 17032-17039, 1992

56. Taylor J M, Jacob-Mosier G G, Lawton R G, VanDort M, and Neubig R R: “Receptor and membrane interaction sites on Gbeta. A receptor-derived peptide binds to the carboxylterminus” J. Biol. Chem. 271, 3336-3339, 1996

57. Kisselev O, Ermolaeva M, and Gautam N: “Efficient interaction with a receptor requires a specific type of prenyl group on the G protein gammα subunit” J. Biol. Chem. 270, 25356-25358, 1995

58. Yasuda H, Lindorfer M A, Woodfork K A, Fletcher J E, and Garrison J C: “Role of the prenyl group on the G protein gammα subunit in coupling trimeric G proteins to A1 adenosine receptors.” J. Biol. Chem. 271, 18588-18595, 1996

59. Richardson M and Robishaw J D: “The alpha2A-adrenergic receptor discriminates between Gi heterotrimers of different betagammα subunit composition in Sf9 insect cell membranes” J. Biol. Chem. 274, 13525-13533, 1999

60. Hou Y, Azpiazu I, Smrcka A, and Gautam N: “Selective role of G protein gamma subunits in receptor interaction” J. Biol. Chem. 275, 38961-38964, 2000

61. Kisselev O G, Meyer C K, Heck M, Ernst O P, and Hofmann K P: “Signal transfer from rhodopsin to the G-protein: evidence for a two-site sequential fit mechanism” Proc.Natl.Acad.Sci.U.S.A 96, 4898-4903, 1999

62. Bluml K, Mutschler E, and Wess I: “Identification of an intracellular tyrosine residue critical for muscarinic receptor-mediated stimulation of phosphatidylinositol hydrolysis” J. Biol. Chem. 269, 402-405, 1994

63. Bluml K, Mutschler E, and Wess J: “Functional role of a cytoplasmic aromatic amino acid in muscarinic receptor-mediated activation of phospholipase C” J. Biol. Chem. 269, 11537-11541, 1994

64. Hill-Eubanks D, Burstein E S, Spalding T A, Brauner-Osborne H, and Brann M R: “Structure of a G-protein-coupling domain of a muscarinic receptor predicted by random saturation mutagenesis” J. Biol. Chem. 271, 3058-3065 , 1996

65. Burstein E S, Spalding T A, and Brann M R: “Constitutive activation of chimeric m2/m5 muscarinic receptors and delineation of G-protein coupling selectivity domains.” Biochem.Pharmacol. 51, 539-544, 1996

66. Burstein E S, Spalding T A, Hill-Eubanks D, and Brann MR: “Structure-function of muscarinic receptor coupling to G proteins. Random saturation mutagenesis identifies a critical determinant of receptor affinity for G proteins” J. Biol .Chem. 270, 3141-3146, 1995

67. Liu J, Blin N, Conklin B R, and Wess J: “Molecular mechanisms involved in muscarinic aetylcholine receptor-mediated G protein activation studied by insertion mutagenesis” J. Biol. Chem. 271, 6172-6178, 1996

68. Burstein E S, Spalding T A, and Brann M R: “The second intracellular loop of the m5 muscarinic receptor is the switch which enables G-protein coupling” J. Biol. Chem. 273, 24322-24327, 1998

69. Yamashita T, Terakita A, and Shichida Y: “Distinct roles of the second and third cytoplasmic loops of bovine rhodopsin in G protein activation” J. Biol. Chem. 275, 34272-34279, 2000

70. Palczewski K, Kumasaka T, Hori T, Behnke C A, Motoshima H, Fox B A, Le T, I, Teller D C, Okada T, Stenkamp RE, Yamamoto M, and Miyano M: “Crystal structure of rhodopsin: A G protein-coupled receptor” Science 289, 739-745, 2000

71. Gether U and Kobilka B K: “G protein-coupled receptors. Ii. Mechanism Of agonist activation” J. Biol. Chem. 273, 17979-17982, 1998

72. Wess J: “Molecular basis of receptor/G-protein-coupling selectivity” Pharmacol.Ther. 80, 231-264, 1998

73. Hulme E C, Lu Z L, Ward S D, Allman K, and Curtis C A: “The conformational switch in 7-transmembrane receptors: the muscarinic receptor paradigm” Eur.J.Pharmacol. 375, 247-260, 1999

74. Sakinar T P: “Rhodopsin: a prototypical G protein-coupled receptor” Prog.Nucleic Acid Res.Mol. Biol. 59, 1-34, 1998

75. Han M, Lin S W, Minkova M, Smith S O, and Sakinar TP: “Functional interaction of transmembrane helices 3 and 6 in rhodopsin. Replacement of phenylalanine 261 by alanine causes reversion of phenotype of a glycine 121 replacement mutant”J. Biol. Chem. 271, 32337-32342, 1996

76. Asano T, Ui M, and Ogasawara N: “Prevention of the agonist binding to yaminobutyric acid B receptors by guanine nucleotides and islet-activating protein, pertussis toxin, in bovine cerebral cortex” J. Biol. Chem. 260, 12653-12658, 1985

77. Pobiner B F, Northup J K, Bauer P H, Fraser E D, and Garrison J C: “The inhibitory GTP-binding regulatory protein, Gi3, can couple angiotensin II receptors to inhibition of adenylyl cyclase in hepatocytes” Mol. Pharmacol. 40, 156-167, 1991

78. Clawges H M, Depree K M, Parker E M, and Graber S G: “Human 5-HT1 receptor subtypes exhibit distinct G protein coupling behaviors in membranes from Sf9 cells.” Biochemistry 36, 12930-12938, 1997

79. Figler R A, Graber S G, Lindorfer M A, Yasuda H, Linden J, and Garrison J C: “Reconstitution of recombinant bovine A1 adenosine receptors in Sf9 cell membranes with recombinant G proteins of defined composition.” Mol.Pharmacol. 50, 1587-1595, 1996

80. Hartman J I and Northup J K: “Functional reconstitution in situ of 5-hydroxytryptamine2c (5HT2c) receptors with αq and inverse agonism of 5HT2c receptor antagonists.” J. Biol. Chem. 271, 22591-22597, 1996

81. Samama P, Cotecchia S, Costa T, and Lefkowitz R J: “A mutation-induced activated state of the β2-adrenergic receptor. Extending the ternary complex model” J. Biol. Chem. 268, 4625-4636, 1993

82. Leff P: “The two-state model of receptor activation” Trends Pharmacol. Sci. 16, 89-97, 1995

83. Scaramellini C and Leff P: “A three-state receptor model: predictions of multiple agonist pharmacology for the same receptor type” Ann.N.Y.Acad.Sci. 861, 97-103, 1998

84. Gether U: “Uncovering molecular mechanisms involved in activation of G protein-coupled receptors” Endocr.Rev. 21, 90-113, 2000

85. Berrie C P, Birdsall N J, Hulme E C, Keen M, and Stockton J M: “Solubilization and characterization of guanine nucleotide-sensitive muscarinic agonist binding sites from rat myocardium.” Br. J. Pharmacol. 82, 853-861, 1984

86. Florio V A and Sternweis P C: “Mechanisms of muscarinic receptor action on Go in reconstituted phospholipid vesicles” J. Biol .Chem. 264, 3909-3915,

87. Parker E M, Grisel D A, Iben L G, Nowak H P, Mahle C D, Yocca F D, and Gaughan GT: “Characterization of human 5-Hydroxytryptamine₁ receptors expressed in Sf9 insect cells” Eur.J.Pharm. 268, 43-53,

88. Coljee V W, Murray H L, Donahue W F, and Jarrell K A: “Seamless gene engineering using RNA- and DNA-overhang cloning” Nat.Biotechnol. 18, 789-791, 2000

The invention claimed:

1. A chimeric α subunit of G proteins, which chimeric α subunit is represented by formula (I): ti B₁—B₂—B₃ (I) PS
wherein
B₁ is the N-terminus and B₃ is the C-terminus of the chimeric α subunit;
B₁ is a peptide having the N-terminal amino acid sequence of n amino acids in length from donor alpha, wherein donor alpha is an α subunit of a donor G protein;
B₃ is a peptide having the C-terminal amino acid sequence of c amino acids in length from donor alpha;
B₂ is selected from the group consisting of (A) recipient alpha, which is an α subunit of a recipient G protein different from the α subunit of the donor G protein, (B) recipient alpha minus n−10, n−9, n−8, n−7, n−6, n−5, n−4, n−3, n−2, n−1, n, n+1, n+2, n+3, n+4, n+5, n+6, n+7, n+8, n+9 or n+10 consecutive amino acid residues from the N-terminus, (C) recipient alpha minus c−10, c−9, c−8, c−7, c−6, c−5, c−4, c−3, c−2, c−1, c, c+1, c+2, c+3, c+4, c+5, c+6, c+7, c+8, c+9 or c+10 consecutive amino acid residues from the C-terminus, and (D) recipient alpha minus n−10, n−9, n−8, n−7, n−6, n−5, n−4, n−3, n−2, n−1, n, n+1, n+2, n+3, n+4, n+5, n+6, n+7, n+8, n+9 or n+10 consecutive amino acid residues from the N-terminus and minus c−10, c−9, c−8, c−7, c−6, c−5, c−4, c−3, c−2, c−1, c, c+1, c+2, c+3, c+4, c+5, c+6, c+7, c+8, c+9 or c+10 consecutive amino acid residues from the C-terminus; wherein
n and c are independently about 3 to about 50, about 3 to about 44, about 3 to about 42, about 3 to about 40, about 3 to about 38, about 3 to about 35, about 6 to about 50, about 6 to about 44, about 6 to about 42, about 6 to about 40, about 6 to about 38, about 6 to about 35, about 6 to about 32, about 6 to about 30, about 8 to about 38, about 8 to about 35, about 8 to about 32, about 8 to about 30, about 10 to about 32, about 10 to about 30, about 10 to about 28, about 10 to about 25, about 12 to about 25 or about 12 to about 20 amino acid residues;
n and c can be the same or different; and
B₁ and B₂, and B₂ and B₃, are linked with a peptide bond.

2. The chimeric α subunit of claim 1, wherein n and c independently are about 6 to about 50, about 6 to about 44, about 6 to about 42, about 6 to about 40, about 6 to about 38, about 6 to about 35, about 6 to about 3 2, about 6 to about 3 0, about 8 to about 38, about 8 to about 35, about 8 to about 32, about 8 to about 30, about 10 to about 32, about 10 to about 30, about 10 to about 28, about 10 to about 25, about 12 to about 25 or about 12 to about 20.

3. The chimeric α subunit of claim 1, wherein n and c independently are about 6 to about 40, about 6 to about 38, about 6 to about 35, about 6 to about 32 or about 6 to about 30.

4. The chimeric α subunit of claim 3, wherein n and c independently are about 6, about 25, about 31, about 35 or about 37.

5. The chimeric α subunit of claim 1, wherein B₂ is the recipient alpha.

6. The chimeric α subunit of claim 1, wherein B₂ is the recipient alpha minus n−10, n−9, n−8, n−7, n−6, n−5, n−4, n−3, n−2, n−1, n, n+1, n+2, n+3, n+4, n+5, n+6, n+7, n+8, n+9 or n+10 amino acid residues from the N-terminus.

7. The chimeric α subunit of claim 1, wherein B₂ is the recipient alpha minus c−10, c−9, c−8, c−7, c−6, c−5, c−4, c−3, c−2, c−1, c, c+1, c+2, c+3, c+4, c+5, c+6, c+7, c+8, c+9 or c+10 amino acid residues from the C-terminus.

8. The chimeric α subunit of claim 1, wherein B₂ is the recipient alpha minus n−10, n−9, n−8, n−7, n−6, n−5, n−4, n−3, n−2, n−1, n, n+1, n+2, n+3, n+4, n+5, n+6, n+7, n+8, n+9 or n+10 amino acid residues from the N-terminus and minus c−10, c−9, c−8, c−7, c−6, c−5, c−4, c−3, c−2, c−1, c, c+1, c+2, c+3, c+4, c+5, c+6, c+7, c+8, c+9 or c+10 amino acid residues from the C-terminus.

9. The chimeric α subunit of claim 1, wherein the donor alpha is Gq and the recipient alpha is Gi1.

10. The chimeric α subunit of claim 1, wherein the donor alpha is Gi1 and the recipient alpha is Gq.

11. The chimeric α subunit of claim 1, wherein n is 6, c is 35 and B₂ is the recipient alpha minus 35 consecutive amino acid residues in the C-terminus.

12. The chimeric α subunit of claim 11, wherein the donor alpha is Gq and the recipient alpha is Gi1.

13. The chimeric α subunit of claim 12 represented by SEQ ID NO:12.

14. The chimeric α subunit of claim 1, wherein n is 37, c is 35 and B₂ is the recipient alpha minus 31 consecutive amino acid residues in the N-terminus and minus 35 consecutive amino acid residues in the C-terminus.

15. The chimeric α subunit of claim 14, wherein the donor alpha is Gq and the recipient alpha is Gi1.

16. The chimeric α subunit of claim 15 represented by SEQ ID NO₄:16.

17. The chimeric α subunit of claim 1, wherein n is 31, c is 25 and B₂ is the recipient alpha minus 37 consecutive amino acid residues in the N-terminus and minus 25 consecutive amino acid residues in the C-terminus.

18. The chimeric α subunit of claim 17, wherein the donor alpha is Gi1 and the 20 recipient alpha is Gq.

19. The chimeric α subunit of claim 18 represented by SEQ ID NO:18.

20. A chimeric G protein comprising a β subunit of a G protein, a γ subunit of a G protein and a chimeric α subunit of claim 1, wherein the β subunit and γ subunit are from the same or different G proteins.

21. A protein having an amino acid sequence with 95% identity with the amino acid sequence of the protein of claim 13 (SEQ ID NO:12), wherein when said protein is combined with a ≈ subunit and a γ subunit of G proteins, the resultant heterotrimeric protein has a receptor coupling specificity similar to that of a heterotrimer formed by the chimeric α subunit represented by SEQ ID NO:12, the P subunit and the y subunit.

22. A protein having an amino acid sequence with 95% identity with the amino acid sequence of the protein of claim 16 (SEQ ID NO:16), wherein when said protein is combined with α subunit and a y subunit of G proteins, the resultant heterotrimeric protein has a receptor coupling specificity similar to that of a heterotrimer formed by the chimeric α subunit represented by SEQ ID NO:16, the β subunit and the γ subunit.

23. A protein having an amino acid sequence with 95% identity with the amino acid sequence of the protein of claim 19 (SEQ ID NO:18), wherein when said protein is combined with a β subunit and a γ subunit of G proteins, the resultant heterotrimeric protein has a receptor coupling specificity similar to that of a heterotrimer formed by the chimeric α subunit represented by SEQ ID NO:18, the β subunit and the γ subunit.

24. A DNA comprising a nucleotide sequence encoding the chimeric α subunit of claim 13, wherein the nucleotide sequence is represented by SEQ ID NO:11.

25. A DNA comprising a nucleotide sequence encoding the chimeric α subunit of claim 16, wherein the nucleotide sequence is represented by SEQ ID NO:15.

26. A DNA comprising a nucleotide sequence encoding the chimeric α subunit of claim 19, wherein the nucleotide sequence is represented by SEQ ID NO:17.

27. A DNA comprising a nucleotide sequence which can hybridize with the nucleotide sequence encoding the chimeric α subunit of claim 13, wherein the nucleotide sequence is represented by SEQ ID NO:11 in a stringency condition of 42° C., 0.2×SSC and 0.1% SDS.

28. A DNA comprising a nucleotide sequence which can hybridize with the nucleotide sequence encoding the chimeric α subunit of claim 16, wherein the nucleotide sequence is represented by SEQ ID NO:15 in a stringency condition of 42° C., 0.2×SSC and 0.1% SDS.

29. A DNA comprising a nucleotide sequence which can hybridize with the nucleotide sequence encoding the chimeric α subunit of claim 19, wherein the nucleotide sequence is represented by SEQ ID NO:17 in a stringency condition of 42° C., 0.2×SSC and 0.1% SDS.
